(19) United States
(12) Patent Application Publication
(10) Pub. No.: US 2003/0125287 A1
(43) Pub. Date: Jul. 3, 2003

(54) Antisense oligonucleotides and methods to induce tumor cell death

(75) Inventors: Ekambar R. Kandimalla, Southboro, MA (US); Sudhir Agrawal, Shrewsbury, MA (US); Sai Latha Shankar, New York, NY (US); Sridhar Mani, Riverdale, NY (US); Bridget Shafit-Zagardo, Merrick, NY (US)

Correspondence Address: HALE AND DORR, LLP, 60 STATE STREET, BOSTON, MA, 02109

(73) Assignee: Hybridon, Inc.

(21) Appl. No.: 10/253,132

(22) Filed: Sep. 24, 2002

Related U.S. Application Data
(60) Provisional application No. 60/324,401, filed on Sep. 24, 2001.

(51) Int. Cl. A61K 48/00; C12Q 1/68; C07H 21/04; C12N 5/02

(52) U.S. Cl. 514/44; 435/6; 435/375; 536/23.5

(57) ABSTRACT

This invention relates to the inhibition and down-regulation of survivin expression. The invention provides methods and antisense oligonucleotides for inhibiting or down-regulating survivin expression in cells and promoting apoptosis and cell necrosis.

### FEDERALLY SPONSORED RESEARCH

This study was supported by a federal grant (Grant R01NS38102) from the National Institutes of Health, and accordingly, the U.S. government may have certain rights in this invention.

### BACKGROUND OF THE INVENTION

1. Field of the Invention

This invention relates to the fields of cell biology, medicine and neoplastic diseases. More specifically, this invention relates to the control of cell proliferation through antisense technology.

2. Summary of the Related Art

Survivin is a member of the inhibitor-of-apoptosis (IAP) family of proteins responsible for inhibiting apoptotic cell death during fetal development (Adida et al. (1998) Am. J. Pathol. 152:41-49). Although survivin is not expressed in terminally differentiated cells, it is significantly increased in human tumors. Survivin has been detected in tumors of the brain, breast, lung, colon, pancreas, prostate, liver, and stomach, but not in low-grade non-Hodgkin's lymphomas (Ambrosini et al. (1997) Nature Med. 3:917-921). It has also been shown that primary neuroblastomas and neuroblastoma cell lines express survivin, and the levels of expression are higher than in colorectal, breast, and lung cancer cell lines (Islam et al. (2000) Oncogene 19:617-623).

Immunohistochemical studies have shown that survivin expression correlates with tumor aggressiveness and poor patient prognosis in neuroblastomas and colorectal carcinomas (Adida et al. (1998) Lancet. 351:882-883; Kawasaki et al. (1998) Cancer Res. 58:5071-5074). In addition, elevated survivin protein has been correlated with reduced cell death in these tumors.

Although previous studies have shown that antisense down-regulation of survivin in non-neuronal tumor lines results in enhanced cell death, survivin has not been used as a target for therapeutic invention of tumors of the nervous system. For example, two recent studies have demonstrated that the inhibition of survivin expression in lung and colon carcinoma cell lines and in HeLa cells reduced survivin expression and resulted in cell death (Olie et al. (2000) Cancer Res. 60:2805-2809; Chen et al. (2000) Neoplasia 2:235-241). However, targeting the down-regulation of survivin to induce cell death (apoptosis) in neuronal cells has not been reported. Thus, there is a long-felt need for therapeutics that induce cell death of brain tumor cells, which may pose particularly difficult challenges.

Aggressive brain tumors circumvent cell death by a number of cellular mechanisms that include overcoming cell cycle check points, re-expression of genes expressed early in fetal development, inhibiting death signals, thereby extending cell viability, and promoting resistance to cytotoxicity induced by radiation and chemotherapy. Anti-apoptotic gene families such as the bcl-2 family and inhibitor-of-apoptosis (IAP) family of proteins are often up-regulated in brain tumors, where their role in blocking apoptosis contributes to the pathogenesis of the tumors (Leaver et al. (1998) J. Neurosurg. 12:539-546; Deininger et al. (1999) Cancer 86:1832-1839; LaCasse et al. (1998) Oncogene 17:3247-3259).

Survivin is a member of the mammalian IAP family of anti-apoptotic proteins. First identified in baculovirus, IAP family members contain one or more copies of a 70 amino acid motif known as the baculovirus IAP repeat (BIR) domain that binds to and inhibits caspase activation (Birmbaum et al. (1994) J. Virol. 68:2521-2528). In humans, there are six IAP family members: XIAP; IAP-1; IAP-2; NAIP; apollon (BRUCE); and survivin(Deveraux et al. (1999) Genes Dev. 13:239-252; Chen et al. (1999) Biochem. Biophys. Res. Commun. 264:847-854; Adida et al. (1998) Am. J. Pathol. 152:41-49; Ambrosini et al. (1997) Nature Med. 3:917-921). XIAP, NAIP, c-IAP-1, and c-IAP-2 contain three BIR domains, while apollon and survivin contain one BIR domain (Deveraux et al. (1999) Genes Dev. 13:239-252; Miller (1999) Trends. Cell. Biol. 9:323-328; Reed et al. (2000) Cell 102:545-548).

With the exception of survivin, IAP family members also have a COOH-terminal RING finger motif. In thymocytes, the RING domain has been shown to be necessary for the ubiquitination as well as proteasome mediated degradation of c-IAP-1 and XIAP (Yang et al. (2000) Science 288:874-877).

In addition to its classification as an IAP family member, survivin is believed to function as a cell cycle regulator. In C. elegans, survivin has been shown to be associated with the mitotic spindle and to partially complement the cytokinesis defect induced by BIR-1 deficiency, suggesting a role in the cell cycle (Fraser et al. (1999) Curr. Biol. 9:292-301).

Survivin expression is increased during the G₂/M phase of the cell cycle, where it is considered essential for the transition through the G₂/M cell cycle checkpoint and normal mitosis (Li et al. (1998) Nature 396:580-584). During mitosis, survivin is associated with the mitotic spindle, and microtubule-binding assays have demonstrated that survivin binds to tubulin (Li et al. (1998) Nature 396:580-584). Survivin also has been shown to bind to cdk4 and to aid in the G₁/S cell cycle transition (Suzuki et al. (2000) Oncogene 19:3225-3234). Further studies have suggested that alternative splice variants of survivin may be transported to the nucleus, which may result in regulation of gene expression during cell cycle transition (Rodriguez et al. (2002) Exp. Cell. Res. 275:44-53).

Comparative genomic hybridization studies have determined that neuroblastomas often have a gain in the distal region of 17q. FISH data demonstrated that the survivin gene, which maps to 17q25, is within the 17q gain region (Islam et al. (2000) Oncogene 19:617-623). Indeed, survivin protein is increased in abundance in neuroblastomas and portends poor prognosis (Adida et al. (1998) Lancet 351:882-883). While survivin down-regulation induced apoptosis in lung and colon carcinomas and HeLa cells (Olie et al. (2000) Cancer Res. 60:2805-2809; Chen et al. (2000) Neoplasia 2:235-241) has been demonstrated, such effects have not been studied for tumors of the nervous system. As mortality is nearly 100% with existing treatments for brain tumors, a great need exists for new, relatively non-toxic therapies for nervous system cancer. Therefore, there is a need to develop an effective therapeutic to down-regulate survivin protein levels in cells of the nervous system, in nervous system cancer cells in particular.

### SUMMARY OF THE INVENTION

The present invention provides new synthetic oligonucleotides and methods for blocking survivin activity in cancer cells of the nervous system.

It has been discovered that antisense oligonucleotides targeted to survivin mRNA down-regulate survivin protein and induce cell death in human nervous system tumor cells. Specifically, it has been determined that these antisense oligonucleotides specifically target survivin mRNA sequences and significantly inhibit expression of survivin protein in human neuroblastoma and oligodendroglioma cells. Treatment with the antisense oligonucleotide resulted in apoptotic death of cells that express survivin. These and other determinations have been exploited to provide the present invention, which includes synthetic oligonucleotides complementary to survivin nucleic acid, and methods of their use.

More specifically, in one aspect, the invention provides synthetic oligonucleotides which are complementary to various regions spanning the survivin gene. In some embodiments, these regions include nucleotide locations 1839-1858, 2867-2886, 3180-3199, 3239-3258, 3248-3267, 4385-4404, 5248-5267, 11432-11451, 11897-11916, 11951-11970, and 12241-12260.

In some embodiments, the oligonucleotides of the invention have about 12-30 nucleotides. In at least some embodiments, the oligonucleotides have about 15-25 nucleotides. In one embodiment, the oligonucleotide is about 20 nucleotides in length.

In some embodiments, the oligonucleotides of the invention comprise at least one modified internucleoside linkage. In certain embodiments, that internucleoside linkage is a phosphorothioate or phosphorodithioate internucleoside linkage.

In some embodiments, the oligonucleotides of the invention comprise at least one 2′-substituted ribonucleoside. In some embodiments, the oligonucleotides comprise at least one modified internucleoside linkage and at least one 2′-substituted ribonucleoside. In certain embodiments, the oligonucleotide comprises at least three 2′ substituted ribonucleosides, or at least four 2′- substituted ribonucleosides. In certain embodiments, the 2′-substituted ribonucleoside is a 2′-alkyl or 2′-O-alkyl ribonucleoside. In certain embodiments, the oligonucleotide comprises at least three contiguous deoxyribonucleotides or deoxyribonucleoside phosphorothioates. In certain embodiments, the oligonucleotide comprises at least four contiguous deoxyribonucleotides or deoxyribonucleoside phosphorothioates.

In particular embodiments, the oligonucleotides of the invention comprise a nucleic acid sequence selected from the group consisting of SEQ ID NOS: 1-11. In specific embodiments, an oligonucleotide of the invention comprises a nucleic acid sequence selected from the group consisting of SEQ ID NOS: 6, 7, and 9.

One aspect of the invention is an oligonucleotide having a nucleic acid sequence that is at least 85% identical to a nucleic acid sequence selected from the group consisting of SEQ ID NOS: 1-11. In particular embodiments, the oligonucleotide has a nucleic acid sequence that is at least 85% identical to a nucleic acid sequence selected from the group consisting of SEQ ID NOS: 6, 7, and 9. In some embodiments, the oligonucleotide has phosphorothioate internucleoside linkages.

Another aspect of the invention is an oligonucleotide having a nucleic acid sequence selected from the group consisting of SEQ ID NOS: 1-11. In certain embodiments, the oligonucleotide has a nucleic acid sequence selected from the group consisting of SEQ ID NOS: 6, 7, and 9. In some embodiments, the oligonucleotide has phosphorothioate internucleoside linkages.

In another aspect, the invention provides a method of enhancing apoptosis in a cell expressing survivin, comprising contacting the cell with an oligonucleotide of the invention, as described above. In certain embodiments, the cell expressing survivin is a cancer cell. In particular embodiments, the cancer cell is a nervous system cancer cell. In specific embodiments, the nervous system cancer cell is a neuroblastoma cell or an oligodendroglioma cell.

In yet another aspect, the invention also provides a method of inhibiting the synthesis of survivin in a cell that expresses survivin, comprising contacting the cell with an oligonucleotide of the invention, as described above.

In still another aspect, the invention provides a method of inhibiting the growth of a cancer cell expressing survivin, comprising contacting the cell with an oligonucleotide of the invention, as described above. In some embodiments, the cancer cell is a nervous system cancer cell, such as a neuroblastoma cell or an oligodendroglioma cell.

In yet another aspect, the invention provides a pharmaceutical composition comprising an antisense oligonucleotide complementary to the survivin mRNA or gene and a pharmaceutically acceptable carrier. The invention also provides a method for treating a nervous system tumor in a mammal. In this method a therapeutically effective amount of a survivin-specific antisense oligonucleotide according to the invention or of a pharmaceutical formulation according to the invention is administered to the mammal. In some embodiments, the mammal is a human. In some embodiments, the nervous system tumor is a neuroblastoma or an oligodendroglioma.

### BRIEF DESCRIPTION OF THE DRAWINGS

The foregoing and other objects of the present invention, the various features thereof, as well as the invention itself, may be more fully understood from the following description, when read together with the accompanying drawings.

FIG. 1A is a representation of a Northern blot showing the expression of the 1.9 kb survivin transcript in five nervous system cancer cell lines, MSN, HTB 14, HTB 17, TC 620, and HOG.

FIG. 1B is a representation of a Western blot showing the expression of the 16.5 kD survivin protein in nervous system tumor cell lines MSN, HTB-14, HTB-17, and HOG.

FIG. 2A is a representation of a Western blot showing elevated survivin expression in MSN cells treated with G₂/M cell cycle checkpoint blockers.

FIG. 2B is a representation of a Western blot showing elevated survivin expression in TC620 oligodendroglioma cells treated with nocadozole.

FIG. 3A is a representation of a Western blot showing down-regulation of survivin protein levels in MSN cancer cells treated with six different survivin antisense oligonucleotides.

FIG. 3B is a representation of a Western blot showing concentration-dependent decrease in survivin protein levels in MSN cells treated with survivin antisense oligonucleotide 903.

FIG. 4A is a graphic representation showing the dose-dependent increase in cell death, as represented by Trypan blue positive cells, following survivin antisense oligonucleotide 904 treatment of MSN cells.

FIG. 4B is a graphic representation showing the increase in cell death, as represented by Trypan blue positive cells, following treatment of MSN cells with survivin antisense oligonucleotide 904 or 906, alone or in combination with the caspase inhibitor zVAD-fmk.

FIG. 4C is a representation of a Western blot showing survivin expression in MSN cells treated with 400 nM or 600 nM survivin antisense oligonucleotide 904.

FIG. 5A is a representation of a Western blot showing dose-dependent decrease in survivin protein expressed in TC620 cancer cells after 48 hours of treatment with different concentrations of survivin antisense oligonucleotides.

FIG. 5B is a representation of a Western blot showing cleavage of PARP in TC620 cells following survivin antisense treatment.

FIG. 6A is a graphic representation showing the dose-dependent increase in cell death in TC620 cells treated with different concentrations of survivin antisense oligonucleotide 904.

FIG. 6B is a graphic representation showing the increase in cell death following treatment of TC620 cells with survivin antisense oligonucleotide 904 or 906 alone, and the subsequent decrease in cell death upon combination treatment with the caspase inhibitor zVAD-fmk.

FIG. 7A is a representation of a photomicrograph showing the nuclear morphology of TC620 cells following treatment with lipofectin and PI staining.

FIG. 7B is a representation of a photomicrograph showing the nuclear morphology of TC620 cells following treatment with 600 nM mismatch oligonucleotide 1132 and PI staining.

FIG. 7C is a representation of a photomicrograph showing the nuclear morphology of TC620 cells following treatment with 600 nM antisense oligonucleotide 904 and PI staining. The arrows point to apoptotic nuclei.

FIG. 7D is a representation of a photomicrograph showing the nuclear morphology of TC620 cells following treatment with 600 nM antisense oligonucleotide 906 and PI staining. The arrows point to abnormal macronuclei that are multilobed.

FIG. 7E is a representation of a photomicrograph showing the nuclear morphology of TC620 cells following treatment with 600 nM antisense oligonucleotide 904 and PI staining. The arrow points to abnormal macronuclei that are multilobed.

FIG. 7F is a representation of a phase micrograph of FIG. 7E showing that the abnormal multilobed nuclei are within individual cells. The arrow points to abnormal macronuclei that are multilobed.

FIG. 8A is a graphic representation showing the changes in nuclear morphology following treatment of TC620 cells with survivin antisense oligonucleotide 904 or 906 alone, or in combination with the caspase inhibitor zVAD-fmk.

FIG. 8B is a graphic representation showing the increase in the number of cells in metaphase following treatment of TC620 cells with survivin antisense oligonucleotide 904 or 906 alone and in combination with the caspase inhibitor zVAD-fmk.

FIG. 9A is a representation of a photomicrograph showing the nuclear morphology of MSN cells following treatment with lipofectin and PI staining.

FIG. 9B is a representation of a photomicrograph showing the nuclear morphology of MSN cells following treatment with 600 nM mismatch oligonucleotide 1132 and PI staining.

FIG. 9C is a representation of a photomicrograph showing the nuclear morphology of MSN cells following treatment with 600 nM antisense oligonucleotide 904 and PI staining. The arrows point to abnormal multiple multilobed nuclei.

FIG. 9D is a representation of a photomicrograph showing the nuclear morphology of MSN cells following treatment with 600 nM antisense oligonucleotide 906 and PI staining. The arrows point to abnormal multiple multilobed nuclei.

FIG. 9E is a representation of a photomicrograph showing the nuclear morphology of MSN cells following treatment with 600 nM antisense oligonucleotide 904 and PI staining. The arrows point to abnormal multiple multilobed nuclei. The arrowhead points to partially condensed nuclei.

FIG. 9F is a representation of a phase micrograph of FIG. 7E showing that the abnormal multilobed nuclei are present within individual cells. The arrows point to abnormal multiple multilobed nuclei. The arrowhead points to partially condensed nuclei.

FIG. 9G is a graphic representation showing the changes in nuclear morphology following treatment of MSN cells with survivin antisense oligonucleotide 904 or 906 alone, or in combination with the caspase inhibitor zVAD-fmk.

FIG. 9H is a representation of a photomicrograph showing MSN cells treated with lipofectin and double-labeled with apoptosis-inducing factor (AIF) and DAPI.

FIG. 9I is a representation of a photomicrograph showing nuclear translocation of AIF in MSN cells treated with 600 nM survivin antisense oligonucleotide 904 and double-labeled with AIF and DAPI.

FIG. 9J is a representation of a photomicrograph showing MSN cells treated with 600 nM mismatch oligonucleotide 1132 and double-labeled with AIF and DAPI.

FIG. 9K is a representation of a photomicrograph showing nuclear translocation of AIF in MSN cells treated with 600 nM survivin antisense oligonucleotide 906 and double-labeled with AIF and DAPI. The scale bar represents 20 μm.

FIG. 10A is a representation of a Western blot showing increased expression of XIAP in MSN cells following treatment with different survivin antisense oligonucleotides.

FIG. 10B is a representation of a Western blot showing increases in expression levels of XIAP following treatment of TC620 oligodendroglioma cells with different concentrations of survivin antisense oligonucleotide 904.

### DETAILED DESCRIPTION

The published patent and scientific literature referred to herein establishes knowledge that is available to those with skill in the art. The issued U.S. patents, published applications, published foreign patent applications, and references, including GenBank database sequences, that are cited herein are hereby incorporated by reference to the same extent as if each were specifically and individually indicated to be incorporated by reference. Any inconsistency between these publications and the present disclosure shall be resolved in favor of the present disclosure.

The invention provides compositions and methods for down-regulating survivin present in human tumor cells by inhibiting its expression at the nucleic acid level. The invention provides for the specific inhibition of the synthesis of survivin protein, which has been determined to be responsible for inhibiting apoptotic cell death of tumor cells, and thus provides a therapeutic treatment for cancer.

The inventors have made the discovery that antisense oligonucleotides targeted to survivin mRNA down-regulate survivin protein and induce cell death in human nervous system tumor cells. Specifically, it has been determined that these antisense oligonucleotides specifically target survivin mRNA and significantly inhibit expression of survivin protein in human neuroblastoma and oligodendroglioma cells. Treatment with the antisense oligonucleotides results in death of cells that express survivin.

These and other determinations have been exploited to provide the present invention, which includes synthetic oligonucleotides complementary to survivin nucleic acid, and methods of their use.

As used herein, the term “oligonucleotide” includes polymers of two or more deoxyribonucleosides, ribonucleosides, or any combination thereof. In some embodiments, such oligonucleotides have from about 6 to about 50 nucleoside residues, in some embodiments from about 12 to about 30 nucleoside residues, and in other embodiments, from about 15 to about 25 nucleoside residues. The nucleoside residues may be coupled to each other by any of the numerous known internucleoside linkages. Such internucleoside linkages include, without limitation, phosphorothioate, phosphorodithioate, alkylphosphonate, alkylphosphonothioate, phosphotriester, phosphoramidate, siloxane, carbonate, carboxymethylester, acetamidate, carbamate, thioether, bridged phosphoramidate, bridged methylene phosphonate, bridged phosphorothioate, and sulfone internucleoside linkages. These internucleoside linkages in at least some embodiments are phosphotriester, phosphorothioate, phosphorodithioate, or phosphoramidate linkages, or combinations thereof. In at least some embodiments, the oligonucleotides of the invention comprise at least one phosphorothioate or phosphorodithioate internucleoside linkage. In particular embodiments, the oligonucleotides of the invention comprise at least one phosphorothioate internucleoside linkage.

Oligonucleotides of the invention can include naturally occurring nucleosides, modified nucleosides, or mixtures thereof. The term “modified nucleoside” refers to a nucleoside that includes a modified heterocyclic base, a modified sugar moiety, or a combination thereof. For example, oligonucleotides of the invention may include 2′-substituted ribonucleosides. For purposes of the invention, the term “2′-substituted ribonucleoside” includes ribonucleosides in which the hydroxyl group at the 2′ position of the pentose moiety is substituted to produce a 2′-O-substituted ribonucleoside. For example, such substitution is with a lower alkyl group containing 1-6 saturated or unsaturated carbon atoms, or with an aryl or allyl group having 2-6 carbon atoms or 6-10 carbon atoms, wherein such alkyl, aryl, or allyl group may be unsubstituted or may be substituted, e.g., with halo, hydroxy, trifluoromethyl, cyano, nitro, acyl, acyloxy, alkoxy, carboxyl, carbalkoxyl, or amino groups. The term “2′-substituted ribonucleoside” also includes ribonucleosides in which the 2′ hydroxyl group is replaced with a lower alkyl group containing 1-6 saturated or unsaturated carbon atoms, or with an amino or halo group.

The term “alkyl,” as employed herein, refers to straight and branched chain aliphatic groups having from 1 to 12 carbon atoms, and in some embodiments 1-8 carbon atoms, and in other embodiments 1-6 carbon atoms, which may be optionally substituted with one, two or three substituents. Unless otherwise apparent from context, the term “alkyl” is meant to include saturated, unsaturated, and partially unsaturated aliphatic groups. When unsaturated groups are particularly intended, the terms “alkenyl” or “alkynyl” will be used. When only saturated groups are intended, the term “saturated alkyl” will be used. In some embodiments, the saturated alkyl groups include, without limitation, methyl, ethyl, propyl, isopropyl, butyl, isobutyl, sec-butyl, tert-butyl, pentyl, and hexyl.

The term “oligonucleotide” also encompasses any other organic base-containing polymer, including, but not limited to, polymers having peptide nucleic acid (PNA), peptide nucleic acid with phosphate groups (PHONA), locked nucleic acid (LNA), or morpholino backbones, and oligonucleotides having backbone sections with allyl linkers or amino linkers.

Also encompassed by the term “oligonucleotide” are polymers having chemically modified bases or sugars and/or having additional substituents including, without limitation, lipophilic groups, intercalating agents, diamines, and adamantane.

The oligonucleotides of the invention are complementary to nucleic acids encoding survivin. For purposes of the invention, the term “complementary” means having the ability to hybridize to a genomic region, a gene, or an RNA transcript thereof, under physiological conditions. Such hybridization is ordinarily the result of base-specific hydrogen bonding between complementary strands, typically to form Watson-Crick or Hoogsteen base pairs, although other modes of hydrogen bonding, as well as base stacking, can lead to hybridization. As a practical matter, such hybridization can be inferred from the observation of specific gene expression inhibition, which may be at the level of transcription or translation (or both). Useful oligonucleotides include chimeric oligonucleotides and hybrid oligonucleotides.

As used herein, a “chimeric oligonucleotide” refers to an oligonucleotide having more than one type of internucleoside linkage. One embodiment of such a chimeric oligonucleotide is an oligonucleotide comprising regions of different internucleoside linkages, such as phosphorothioate, phosphorodithioate, and phosphodiester linkages, the regions in some embodiments comprising from about 2 to about 12 nucleosides. In some embodiments, useful chimeric oligonucleotides contain at least one, or in some embodiments, at least three or four consecutive internucleoside linkages that are phosphodiester or phosphorothioate linkages, or combinations thereof. Some useful oligonucleotides of the invention have an alkylphosphonate-linked region or an alkylphosphonothioate-linked region (see e.g., U.S. Pat. Nos. 5,635,377 and 5,366,878). Inverted chimeric oligonucleotides are also contemplated, as described in U.S. Pat. Nos. 5,652,356, 5,973,136, and 5,773,601.

For purposes of the invention, a “hybrid oligonucleotide” refers to an oligonucleotide having more than one type of nucleoside. One embodiment of such a hybrid oligonucleotide comprises a ribonucleoside or 2′-O-substituted ribonucleoside region, in at least some embodiments comprising from about 2 to about 12 2′-O-substituted nucleosides, and a deoxyribonucleoside region. In some embodiments, such a hybrid oligonucleotide contains at least three consecutive deoxyribonucleosides and contains ribonucleosides, 2′-O-substituted ribonucleosides, or combinations thereof (see e.g., Metelev and Agrawal, U.S. Pat. Nos. 5,652,355 and 5,652,356). Inverted hybrid oligonucleotides are also contemplated as described in U.S. Pat. No. 5,652,356.

In some embodiments, oligonucleotides of the invention are mixed backbone oligonucleotides (MBOs), which contain centrally-modified or end-modified nucleosides with appropriately placed segments of modified internucleoside linkages, such as phosphorothioates, methylphosphonates, phosphodiesters and segments of modified oligodeoxy- or oligoribo-nucleotides (Agrawal (1997) Proc. Natl. Acad. Sci. (USA) 94: 2620-2625; Agrawal (1999) Biochem. Biophys. Acta 1489: 53-67).

The terms “neoplastic cell” and “cancer cell” are used to denote a cell that shows aberrant cell growth. In at least some embodiments, the aberrant cell growth of a neoplastic cell is increased cell growth. A neoplastic cell may be a hyperplastic cell, a cell that shows a lack of contact inhibition of growth in vitro, a benign tumor cell that is incapable of metastasis in vivo, or a cancer cell that is capable of metastases in vivo and that may recur after attempted removal. The term “tumorigenesis” is used to denote the induction of cell proliferation that leads to the development of a neoplastic or cancerous growth. Such an assessment of cancer cell growth or proliferation can be made by counting contacted and non-contacted cells using, e.g., a Coulter Cell Counter (Coulter, Miami, Fla.) or a hemacytometer. Where the cells are in a solid growth (e.g., a solid tumor or organ), such an assessment of cell proliferation can be made by measuring the growth with calipers, and comparing the size of the growth of contacted cells with non-contacted cells.

As used herein, the term “necrosed cell” includes dead cells that have undergone programmed cell death, i.e., apoptosis, and cells that test positive when stained with Trypan blue stain.

The term “inhibition of cell proliferation” includes a reduction in the number or size of contacted cells, as compared to non-contacted cells. Thus, a survivin antisense oligonucleotide of the invention that inhibits cell proliferation in a contacted cell may induce the contacted cell to undergo growth retardation, growth arrest, programmed cell death (i.e., to apoptosis), or necrotic cell death.

The synthesis of oligonucleotides according to the invention may be routinely accomplished through any known method. See e.g., Methods in Molecular Biology, Vol 20: Protocols for Oligonucleotides and Analogs pp. 165-189 (S. Agrawal, Ed., Humana Press, 1993); Oligonucleotides and Analogues: A Practical Approach, pp. 87-108 (F. Eckstein, Ed., 1991); Agrawal and Iyer (1995) Curr. Op. in Biotech. 6:12; and Antisense Research and Applications (Crooke and Lebleu, Eds., CRC Press, Boca Raton, 1993). Some well-known synthetic approaches include phosphodiester and phosphotriester chemistries (Khorana et al. (1972) J. Molec. Biol. 72:209, discloses phosphodiester chemistry for oligonucleotide synthesis; Reese (1978) Tetrahedron Lett. 34:3143-3179, discloses phosphotriester chemistry for synthesis of oligonucleotides and polynucleotides).

Additional chemical methods include phosphoramidite and H-phosphonate approaches to synthesis (Beaucage and Caruthers (1981) Tetrahedron Lett. 22:1859-1862 discloses the use of deoxynucleoside phosphoramidites in polynucleotide synthesis; Agrawal and Zamecnik, U.S. Pat. No. 5,149,798 (1992), discloses optimized synthesis of oligonucleotides by the H-phosphonate approach).

The preparation of modified oligonucleotides having a wide variety of modified internucleoside linkages is well-known in the art. For example, Agrawal and Goodchild (1987) Tetrahedron Lett. 28:3539-3542, teaches synthesis of oligonucleotide methylphosphonates using phosphoramidite chemistry. Connolly et al. (1984) Biochemistry 23:3443, discloses synthesis of oligonucleotide phosphorothioates using phosphoramidite chemistry. Jager et al. (1988) Biochemistry 27:7237, discloses synthesis of oligonucleotide phosphoramidates using phosphoramidite chemistry. Agrawal et al. (1988) Proc. Natl. Acad. Sci. USA 85:7079-7083, discloses synthesis of oligonucleotide phosphoramidates and phosphorothioates using H-phosphonate chemistry.

The synthesis of phosphorothioate or mixed backbone modified antisense oligonucleotides targeting different regions of the human survivin mRNA can be performed as described in Agrawal (1997) Proc. Natl. Acad. Sci. (USA) 94:2620-2625. Once synthesized, the oligonucleotides may be placed on any suitable solid support used for solid phase oligonucleotide synthesis, such as controlled-pore glass)(see, e.g., Pon (1993) Meth. Molec. Biol. 20:465-496).

To verify survivin expression in tumors derived from the human nervous system, Northern and Western blot analyses were performed. All of the brain tumor cell lines that were examined expressed survivin mRNA and protein. Higher survivin expression was found in the oligodendroglioma cell lines, while the neuroblastoma, glioblastoma, and astrocytoma cells showed comparable survivin expression. Thus, the presence and abundance of survivin in the nervous system tumor cells is indicative of a role for survivin as a regulator of nervous system tumor survival and pathogenesis.

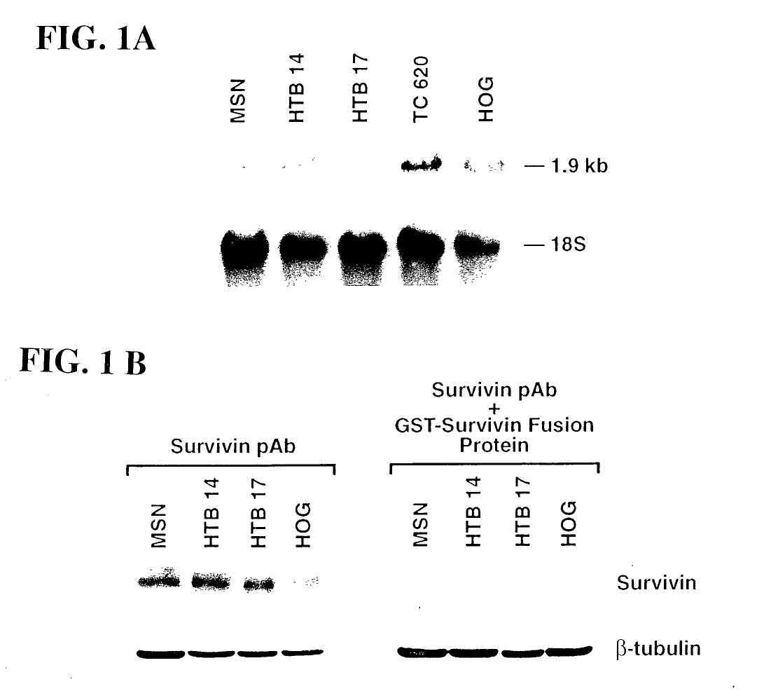

RNA and protein were isolated from cell lines derived from a human neuroblastoma (MSN) (Reynolds et al. (1986) J. Natl. Cancer. Inst. 76:375-387), two oligodendrogliomas (HOG and TC620), an astrocytoma (ATCC No. HTB14, American Type Culture Collection, Manassas, Va.), and a glioblastoma (ATCC No. HTB17, American Type Culture Collection, Manassas, Va.). Northern blot analysis revealed the expression of the 1.9 kb survivin transcript in the five human nervous system tumor cell lines examined (FIG. 1A). Densitometry was used to scan the blots. Twenty jug of total RNA were run on a 1% agarose-formaldehyde gel and transferred to nitrocellulose. The blot was first hybridized with a cDNA probe to the entire coding region of the survivin gene and subsequently hybridized with a cDNA to 18S. The survivin RNA expression was normalized to the expression of 18S RNA. The relative amounts were MSN, 0.075; HTB-14, 0.11; HTB-17, 0.065; TC620, 0.25; HOG, 0.25.

When normalized to 18S RNA, higher expression of survivin was found in the two oligodendroglioma cell lines, while the neuroblastoma, glioblastoma, and astrocytoma showed comparable survivin expression. Immunoblotting confirmed the presence of the 16.5 kD survivin protein in all brain tumor lines (FIG. 1B).

Total protein was isolated from MSN, HTB 14, HTB 17, and HOG cells. 75 μg of total protein was loaded in each lane. The blots were cut at 32.9 kD and the top blot was incubated with a β-tubulin monoclonal antibody (mAb) (1:1000) to confirm equal loading. The bottom blots retaining proteins below 32.9 kD were incubated with a survivin polyclonal antibody (1:500) or with the survivin polyclonal antibody (1:500) pretreated with 10 μg of GST-survivin fusion protein. Visualization was by enhanced chemiluminescence. Blots were scanned in the linear range and data was presented as a ratio of survivin over tubulin in each cell type. The relative amounts were MSN, 0.35; HTB-14, 0.45; HTB-17, 0.39; HOG, 0.13.

When normalized to β-tubulin, survivin protein levels were 3-fold higher in the HTB-14, MSN, and HTB-17 homogenates relative to the HOG cell homogenate. Examination of two additional neuroblastomas (IMR32, ATCC No. CCL-127, American Type Culture Collection, Manassas, Va.) and SK-N-SH, ATCC No. HTB-11, American Type Culture Collection, Manassas, Va.)) and the oligodendroglioma (TC620) also confirmed the presence of survivin. To verify the specificity of the survivin antibody, identical protein blots were incubated with the survivin antibody pre-incubated with the GST-survivin fusion protein. As shown in FIG. 1B, absorption of the antibody eliminated survivin immunoreactivity.

Next, the regulation of survivin expression at G₂/M cell cycle checkpoints was evaluated. First, survivin expression was increased following treatment with the G₂/M checkpoint blockers, vinblastine, nocodazole, and taxotere. Flow cytometry of MSN cells treated with vinblastine and taxotere confirmed that the cells were blocked at the G₂/M cell cycle phase.

Total protein was isolated from MSN cells either untreated or treated with vinblastine (250 nM), nocadozole (10 μM), or taxotere (1 μM). DMSO and ethanol (EtOH) were added as carriers. Each lane had 75 μg of total protein. The blots were cut at 32.9 kD, and the bottom blots were incubated with a survivin polyclonal antibody (1:500), while the top blot was incubated with a β-tubulin mAb (1:1000). In each of the treatments, the fold increase of survivin protein relative to DMSO or EtOH was vinblastine, 1.9; nocadozole, 2.6; taxotere, 1.7.

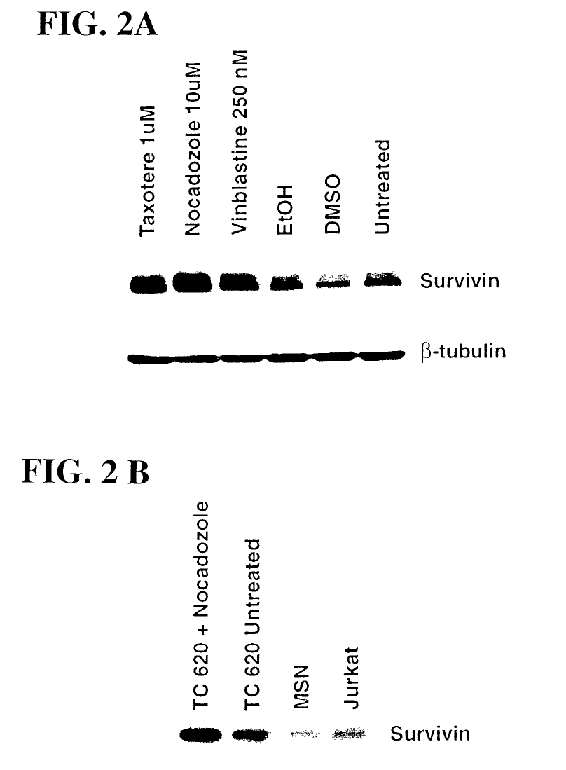

As shown in FIG. 2A, a 1.7-to 2.6-fold increase in survivin protein was observed in MSN total cell lysates treated with the three blockers relative to the DMSO control. A similar 1.6-fold increase in survivin protein was observed in the nocodazole-treated TC620 cells (FIG. 2B), demonstrating that in nervous system tumor cell lines, survivin expression is increased in a G₂/M cell cycle phase-dependent manner. In contrast, cells treated with agents, such as flavopiridol, that typically block cells in G₁/S (Carlson et al. (1996) Cancer Res. 56:2973-2978) did not alter survivin protein abundance. The Western blot as represented in FIG. 2B was prepared by isolating total protein from nocadozole-treated (10 μM, 24 h) and untreated TC620 cells, MSN cells, and Jurkat cells. Each lane had 75 μg of total protein.

To determine whether the inhibition of survivin was sufficient to induce cell death in nervous system tumors, eleven antisense oligonucleotides spanning the survivin gene were analyzed in MSN and TC620 cell lines. The sequences of survivin antisense oligonucleotides are shown in Table 1 as SEQ ID NOS: 1-11. As mentioned above, the oligonucleotides according to the invention are complementary to regions of mRNA that encode at least a portion of survivin. The sequence of survivin mRNA is known (GenBank accession no. U75285). Oligonucleotides of the invention were designed based on the selection criteria described in Agrawal and Kandimalla (2000) Mol. Med. Today 6:72-81.

In some instances, oligonucleotides of the invention have a nucleic acid sequence selected from the group consisting of SEQ ID NOS: 1, 2, 3, 4, 5, 6, 7, 8, 9, 10, and 11. In other instances, the inventive oligonucleotides have a nucleic acid sequence that is at least 85% identical to a nucleic acid sequence selected from the group consisting of SEQ ID NOS: 1, 2, 3, 4, 5, 6, 7, 8, 9, 10, and 11. As used herein, a nucleic acid sequence having a given percent identity to a reference nucleic acid sequence is a nucleic acid sequence in which the number of nucleosides is the same as in the reference sequence, but one or more nucleoside substitutions, most often conservative modifications, has been effected. In some instances, an oligonucleotide of the invention has a nucleic acid sequence that is at least 90% identical to a nucleic acid sequence selected from the group consisting of SEQ ID NOS: 1, 2, 3, 4, 5, 6, 7, 8, 9, 10, and 11. In other instances, the percent identity is at least 93%, for example, at least 95% identical to a nucleic acid sequence selected from the group consisting of SEQ ID NOS: 1, 2, 3, 4, 5, 6, 7, 8, 9, 10, and 11. In yet other instances, the percent identity is at least 97%, for example, at least 98%, or at least 99% identical to a nucleic acid sequence selected from the group consisting of SEQ ID NOS: 1, 2, 3, 4, 5, 6, 7, 8, 9, 10, and 11.

TABLE 1

| | | Location of | |
|---|---|---|---|
| | SEQ ID | complementary | Antisense sequence |
| Oligo | NO: | survivin mRNA | (5′ to 3′) |
| 899 | 1 | 1839-1858 | d(GGCATCACATCCACTCACTT) |
| 900 | 2 | 2867-2886 | d(GCCAGTTCTTGAATGTAGAG) |
| 901 | 3 | 3180-3199 | d(CAGTGGATGAAGGCAGCCTC) |
| 1130 | 4 | 3239-3258 | d(CTTCCAGCTCCTTGAAGCAG) |
| 902 | 5 | 3248-3267 | d(GCTCCCAGCCTTCCAGCTCC) |
| 903 | 6 | 4385-4404 | d(CCCTAGCTCACACTCTCATT) |
| 904 | 7 | 5248-5267 | d(TCTTGGCTCTTTCTCTGTCC) |
| 905 | 8 | 11432-11451 | d(GAGCCTTCCTCTTCATGTCC) |
| 906 | 9 | 11897-11916 | d(GCTTCCCAGTCACATCCTGT) |
| 907 | 10 | 11951-11970 | d(TGTTGGTTTCCTTTGCCTGG) |
| 908 | 11 | 12241-12260 | d(GCCACTGTTACCAGCACAC) |
| 1131 | 12 | Mismatched | d(GCACCTAGCTTTCTAGCCCC) |
| | | Oligo1 | |
| 1132 | 13 | Mismatched | d(GCACCTAGTCTCCCTGCACC) |
| | | Oligo2 | |

As listed above in TABLE 1, eleven oligonucleotides, all of which are 20-mer phosphorothioate survivin antisense oligonucleotides, and which are directed to different regions of survivin mRNA, were designed and are set forth in the Sequence Listing as SEQ ID NOS: 1-11. In addition, to serve as the control, two mismatched oligonucleotides which are not complementary to survivin mRNA were also designed and are set forth in the Sequence Listing as SEQ ID NOS: 12 and 13.

Six survivin (900-906) antisense oligonucleotides were administered at 400 nM to MSN cells in the presence of lipofectin for 48 hours. Each lane has 100 μg of protein. The blots were cut at 32.9 kD and the bottom incubated with the survivin polyclonal antibody (1:500), while the top was incubated with β-tubulin. With survivin antisense oligonucleotide treatment, the percentage of survivin protein over β-tubulin relative to lipofectin was: 900, 77.7%; 901, 38.3%; 903, 24.3%; 904, 35.7%; 905, 55.6w; 906, 26.9%.

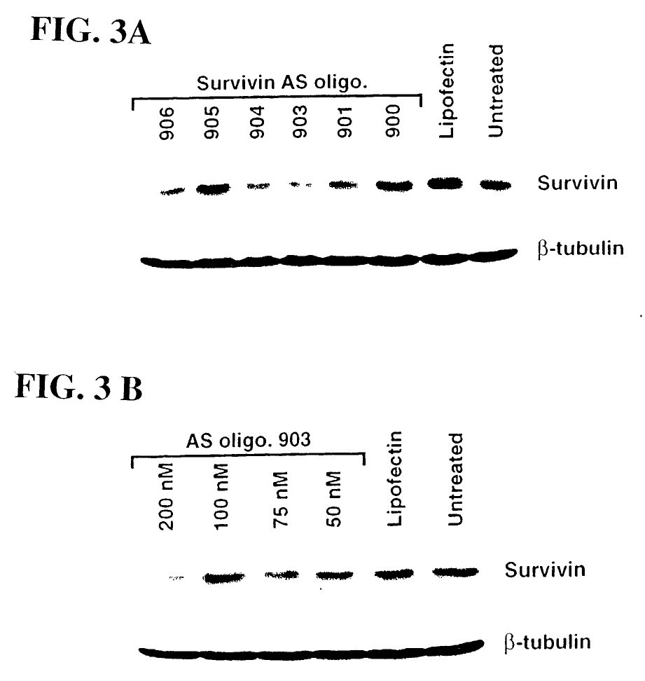

As shown in FIG. 3A, antisense oligonucleotides 903, 904, and 906 (400 nM) were most effective in decreasing survivin protein levels in MSN cells by 76%, 64%, and 73% respectively relative to the lipofectin-treated control following normalization to β-tubulin by densitometry.

Since survivin antisense oligonucleotide 903 decreased survivin protein levels by 76%, the dose dependency of 903 on survivin protein levels was further studied. As expected, mismatched oligonucleotides had no effect on the relative abundance of survivin. As shown in FIG. 3B, a concentration-dependent decrease in survivin protein was observed in MSN cells 48 hours post-treatment with antisense oligonucleotide 903. A 51% reduction in survivin was observed at 200 nM. In the presence of lipofectin, survivin antisense oligonucleotide 903 was administered to MSN cells for 48 hours at 50 nM to 200 nM. Total protein (100 μg) was loaded/lane. With survivin antisense oligonucleotide 903 treatment, the percentage of survivin protein over β-tubulin relative to lipofectin was: 50 nM, 83.9%; 75 nM, 62.6%; 100 nM, 95.6%; 200 nM, 48.5%.

Antisense oligonucleotide 904 was also effective in reducing survivin protein levels in a concentration-dependent manner, while the mismatched oligonucleotide 1132 had no effect. In other experiments using a 100 nM antisense oligonucleotide concentration, a greater reduction in survivin protein levels was observed than is shown in FIG. 3B.

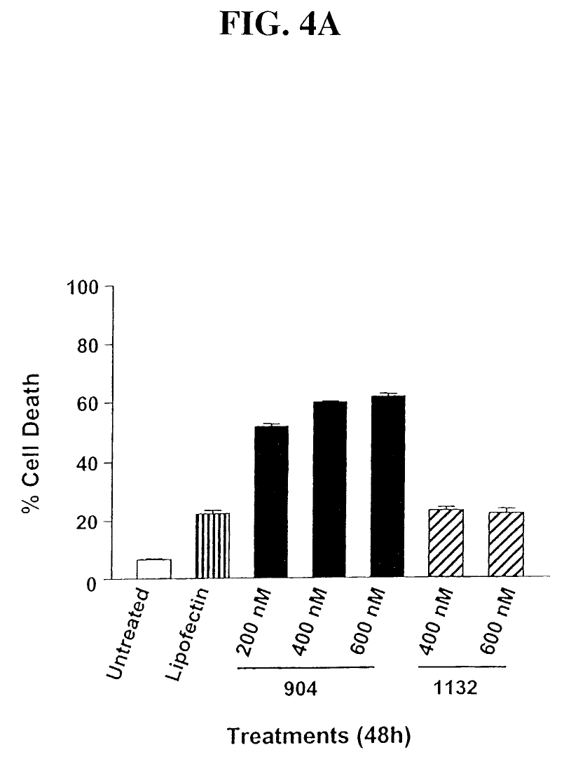

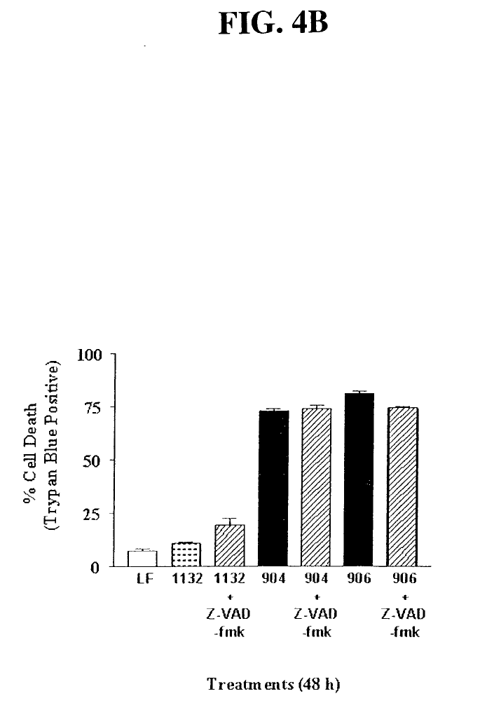

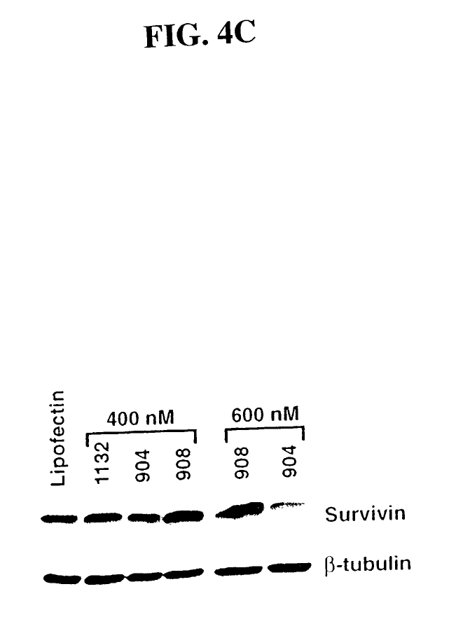

The role of survivin as an inhibitor of apoptosis and as a survival protein for tumors was investigated by examining whether the down-regulation of survivin was sufficient to induce cell death in MSN cells. Following transfection with lipofectin, different concentrations of survivin antisense oligonucleotide 904 (200 nM, 400 nM, 600 nM), or the mismatched oligonucleotide 1132 (400 nM and 600 nM), the number of Trypan blue positive cells (only dead cells are stained by the Trypan blue stain) were counted 48 hours after treatment. The number of dead cells was assessed relative to the lipofectin-treated control cells. In the presence of 200 nM, 400 nM, and 600 nM concentrations of antisense oligonucleotide 904, the percentage of cells that were Trypan blue positive were 52%, 59% and 62%, respectively (FIG. 4A). The mismatched oligonucleotide 1132 (400 nM and 600 nM) was similar to the lipofectin control treatment, wherein 23% (400 nM) and 22% (600 nM) of the cells were Trypan blue-positive. About 6% of the cells in the untreated control were Trypan blue positive.

In another experiment, following transfection with lipofectin, mismatched oligonucleotide 1132 (600 nM), or survivin antisense oligonucleotide 904 or 906 (600 nM), the numbers of Trypan blue positive cells (dead cells) were counted 48 h post-treatment. In the presence of 600 nM survivin antisense oligonucleotide 904 and 906, the percentages of cells that were Trypan blue positive were 73% and 81%, respectively (FIG. 4B). Upon treatment with lipofectin or the mismatched oligonucleotide 1132, only 7-11% of the cells were Trypan blue positive.

The above data demonstrate that the inhibition of survivin following antisense oligonucleotide treatment is sufficient to induce cell death in MSN cells.

FIG. 4C demonstrates that 400 nM and 600 nM survivin antisense treatment decreased survivin protein levels while the mismatched oligonucleotide 1132 did not alter survivin protein levels. Survivin antisense oligonucleotide 904 decreased survivin protein levels by 46% and 60% while survivin levels were unchanged in cells incubated with the mismatched oligonucleotide 1132. MSN cells were treated with lipofectin, survivin antisense oligonucleotide 904 or 908 at 400 nM and 600 nM, or mismatched oligonucleotide 1132 at 400 nM concentrations. Total protein (100 μg) was separated by SDS polyacrylamide gel electrophoresis and transferred to a nitrocellulose support. The blots were cut at 32.9 kD and the bottom blots incubated with the survivin polyclonal antibody (1:500), while the top blots were incubated with tubulin.

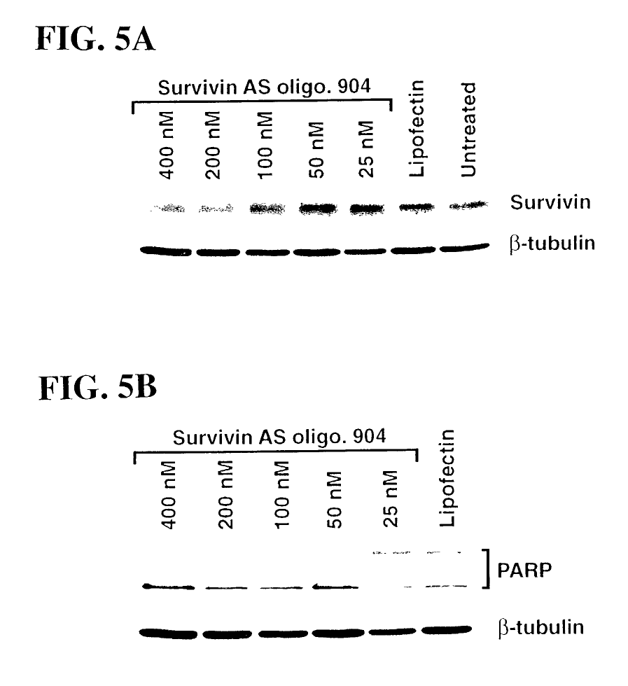

The antisense oligonucleotides having SEQ ID NOS: 1-11 were evaluated for the ability to induce apoptotic cell death in human oligodendroglioma (TC620) cells. It was observed that transfection of TC620 cells with antisense oligonucleotide 904 induced a marked concentration-dependent reduction in survivin protein levels. As shown in FIG. 5A, at a concentration of 400 nM of antisense oligonucleotide 904, there was a 54% decrease in survivin protein abundance relative to lipofectin-treated cells.

Subconfluent TC620 oligodendroglioma cells were transfected with lipofectin or increasing concentrations of survivin antisense oligonucleotide 904 (25 nM to 400 nM). 75 pg of total protein was loaded per lane. Blots were cut at 32.9 kD and the bottom was incubated with a survivin polyclonal antibody (1:500), while the top was incubated with a β-tubulin monoclonal antibody (1:1000). The percentage of survivin protein over β-tubulin relative to lipofectin upon treatment with survivin antisense oligonucleotide 904 was: 25 nM, 109.1%; 50 nM, 97.4%; 100 nM, 67.6%; 200 nM, 45.2%; 400 nM, 45.7%.

To determine whether the decrease in the survivin protein levels in the TC620 cells was associated with a caspase-dependent, apoptotic mode of cell death, PARP (poly(ADP-ribose) polymerase) (Pharmingen, San Diego, Calif.) cleavage was examined by immunoblotting following transfection with varying concentrations of the antisense oligonucleotide 904. As appreciated by one of skill in the art, PARP is a substrate of caspase-3 and is an enzyme that is useful as a positive control for Western blot analysis of ribosylated proteins. 75 μg of total protein was loaded per lane. Following gel electrophoresis and transfer to nitrocellulose membrane, the blot was incubated with a PARP monoclonal antibody (1:500) and visualized by enhanced chemiluminescence.

As shown in FIG. 5B, antisense treatment induced PARP cleavage and generated the 85 kD fragment characteristic of apoptosis. In addition, at concentrations of 100 nM to 400 nM of antisense oligonucleotide 904, a dramatic decrease of the 116 kD PARP protein was detected, and the presence of the 85 kD cleaved fragment was increased relative to the cleaved fragment seen in the lipofectin-treated cells and untreated cells. PARP cleavage in the lipofectin-treated and untreated cells reflects the basal level of spontaneous apoptosis (Yang et al. (2000) Science 288:874-877) in the TC620 cells prior to survivin antisense treatment. These experiments demonstrate that inhibition of survivin expression with antisense oligonucleotide treatment is sufficient to induce apoptotic cell death.

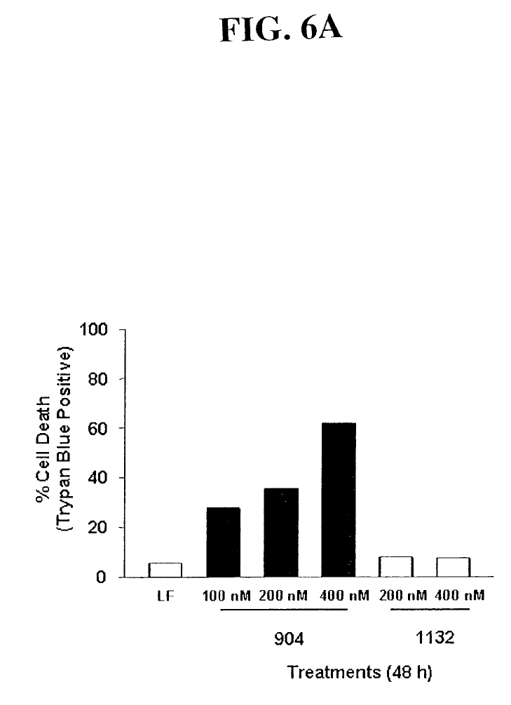

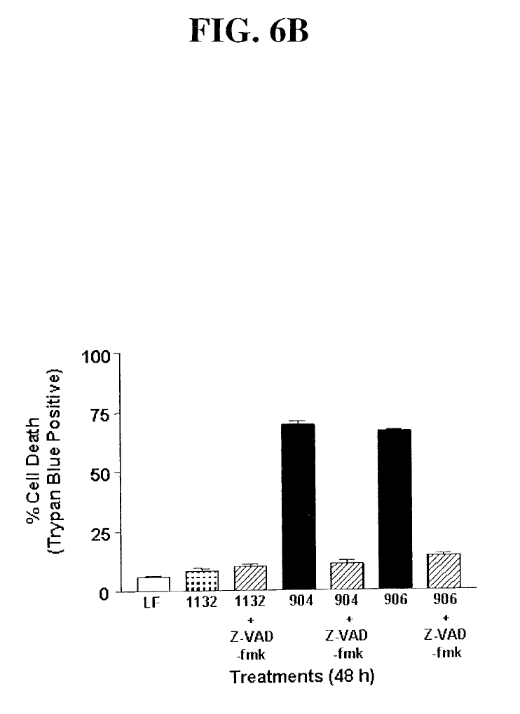

Trypan blue retention assay results confirmed that survivin antisense oligonucleotide 904 induced a concentration-dependent increase in Trypan blue-positive (dead) cells after 48 hours of treatment (FIG. 6A). At 100 nM, 200 nM, and 400 nM, the percentage of dead cells was 28%, 36%, and 62%, respectively. The percentages of Trypan blue-positive cells treated with 200 nM (8%) and 400 nM (7.5%) mismatched oligonucleotide 1132 were similar to the lipofectin control (6%). Cells were treated with lipofectin, survivin antisense oligonucleotide 904 at 100, 200, or 400 nM, or mismatched oligonucleotide 1132 at 200 nM or 400 nM concentrations. Cells were harvested 48 hours after the treatment and stained with 0.04% Trypan blue as described for FIG. 4.

To further examine whether cell death induced by treatment of TC620 cells with survivin antisense oligonucleotides occurs via a caspase-dependent mechanism, another experiment was performed to investigate the effect of a caspase inhibitor on cell death. As shown in FIG. 6B, the percentages of Trypan blue positive cells treated with 600 nM survivin antisense oligonucleotide 904 or 906 were 70% and 67%, respectively. The caspase inhibitor z-Val-Ala-Asp(Ome)-fluoromethyl ketone (zVAD-fmk) effectively decreased the numbers of Trypan blue positive cells induced by survivin antisense oligonucleotide treatment to 11% and 15%, further supporting a caspase-dependent mechanism of apoptotic cell death in the survivin antisense oligonucleotide-treated TC620 cells. TC620 cells were treated with lipofectin or 600 nM survivin antisense oligonucleotide 904, 906, or mismatch oligonucleotide 1132 alone, or co-treated with 20 μM zVAD-fmk.

To confirm the results of the PARP data that survivin down-regulation induced apoptosis, a TUNEL assay was performed. The TUNEL reaction preferentially labels cleaved genomic DNA generated during apoptosis, by the addition of fluorescein dUTP at strand breaks. The TUNEL assay was performed on lipofectin, mismatch oligonucleotide 1132, and survivin antisense oligonucleotide-treated TC620 cells. The percentages of TUNEL-positive cells treated with survivin antisense oligonucleotides 904 or 906 at 400 nM concentration were 52% and 54%, respectively. At 600 nM, 63% of survivin antisense oligonucleotide 904- and 906-treated TC620 cells were TUNEL-positive. Only 4% of the cells were TUNEL-positive in the presence of lipofectin or mismatch oligonucleotide 1132.

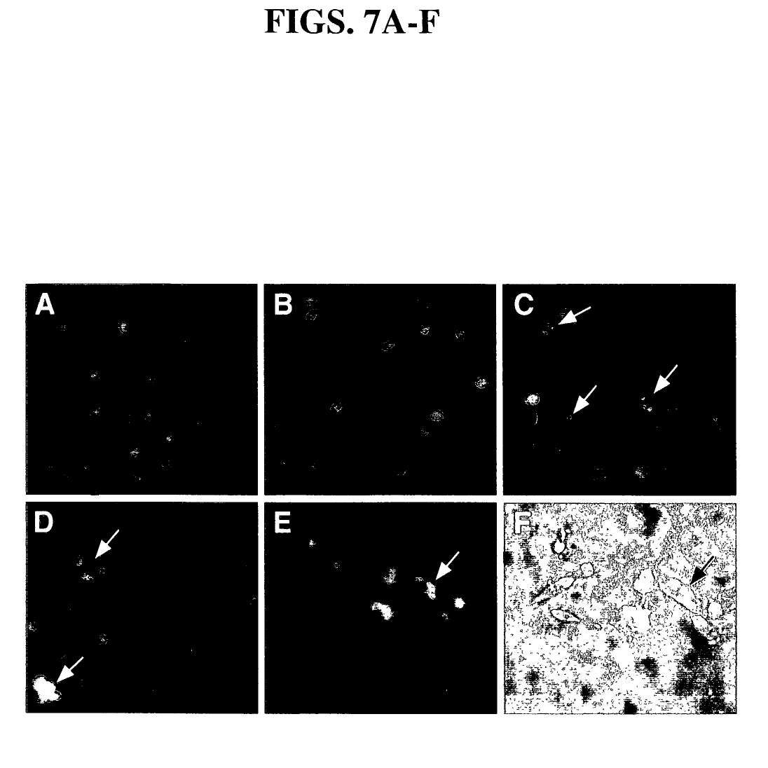

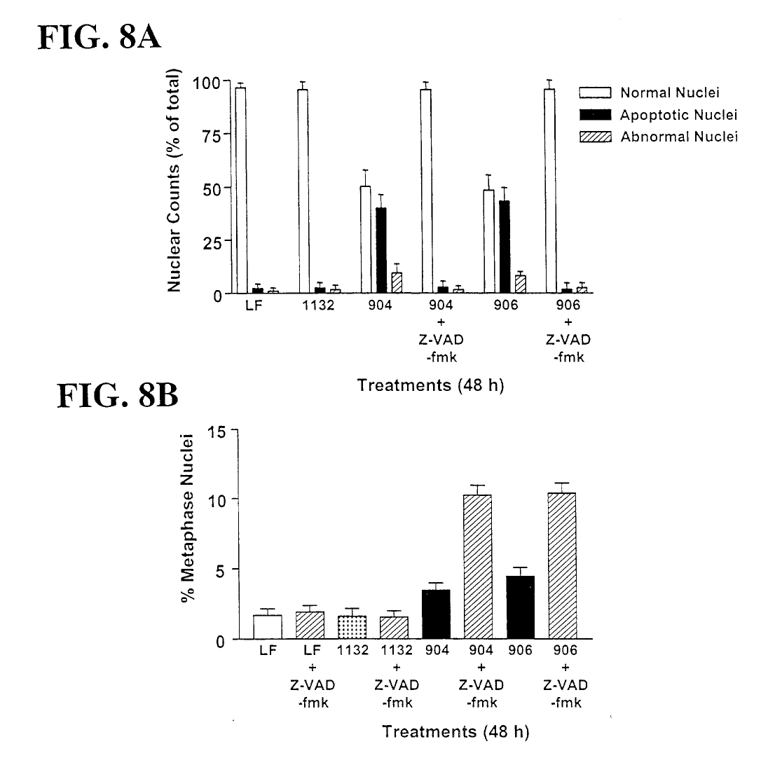

Propidium iodide (PI) staining was performed to investigate changes in nuclear morphology caused by treatment of TC620 cells with survivin antisense oligonucleotides. PI staining demonstrates abnormal nuclear morphology of TC620 cells following survivin antisense oligonucleotide treatment. TC620 cells were treated with lipofectin (FIG. 7A), or 600 nM mismatch oligonucleotide 1132 (FIG. 7B), survivin antisense oligonucleotide 904 (FIGS. 7C and 7E), or 906 (FIG. 7D), and stained with PI. Quantification results for TC620 cells treated with 600 nM survivin antisense oligonucleotide 904, 906, or mismatch oligonucleotide 1132, alone or in the presence of zVAD-fmk, are shown in FIGS. 8A-B. Cells in metaphase were identified as those with chromosomes aligned on the metaphase plate.

PI staining of lipofectin- or mismatch oligonucleotide 1132-treated TC620 cells showed normal nuclear morphology (FIGS. 7A and 7B), with very few apoptotic cells (2%) or cells with abnormal nuclei (1%; FIG. 8A). By contrast, 40-43% of the survivin antisense oligonucleotide-treated TC620 cells revealed nuclei with chromatin fragmentation and apoptotic bodies characteristic of an apoptotic mode of cell death (FIG. 7C, arrows; FIG. 8A). In addition, 9% of the survivin antisense oligonucleotide-treated cells exhibited multiple multilobed nuclei (FIGS. 7D and 7E, arrows; FIG. 8A). Thus, 49-52% of the survivin antisense oligonucleotide-treated TC620 cells had abnormal nuclei. Co-treatment with survivin antisense oligonucleotide and zVAD-fmk dramatically inhibited apoptosis and the abnormal nuclear morphology (FIG. 8A), indicating that down-regulation of survivin results in caspase activation and apoptosis.

Taken together, the results with respect to the 85-kDa PARP cleavage product, PI staining of apoptotic bodies, zVAD-fmk inhibition of cell death, and TUNEL staining of apoptotic cells established apoptotic cell death in the survivin down-regulated TC620 cells.

Co-administration of survivin antisense oligonucleotide and the caspase inhibitor zVAD-fmk decreased the numbers of abnormal nuclei from greater than 40% to 2% (FIG. 8A). Microscopic assessment of the survivin antisense oligonucleotide plus zVAD-fmk-treated cells demonstrated an approximate two-fold increase in the number of cells in metaphase compared with cells treated with survivin antisense oligonucleotide 904 or 906 alone (FIG. 8B). zVAD-fmk in the presence of lipofection and mismatch 1132 did not affect the number of cells in metaphase. This suggests that in the absence of survivin, survivin antisense oligonucleotide-treated TC620 cells cannot complete the normal mitotic cycle, arrest in metaphase, and subsequently undergo apoptosis as a result of mitotic catastrophe. These results support an interplay between mitotic regulation, tumor survival, and cell death.

To determine whether MSN cell death by survivin antisense oligonucleotides was mediated by caspase-3 activation, studies were conducted to examine whether PARP was cleaved following survivin antisense treatment. Immunoblotting of MSN protein homogenates with a PARP-specific antibody failed to detect PARP cleavage following survivin antisense treatment. Since no PARP cleavage was detected, further tests were conducted to determine whether survivin antisense-treated cells have any active caspase-3.

Previous studies have demonstrated that MSN cells activate caspase-3 following staurosporine treatment, and when caspase-3 is activated, PARP is cleaved. As shown in TABLE 2 below, an over 7-fold increase in caspase-3 activity was observed upon treatment of MSN cells with staurosporine; however, MSN cells transfected with survivin antisense oligonucleotides 903 or 904 (400 nM) or the mismatched oligonucleotide 1132 for 48 hours showed no significant increase in caspase-3 activity.

TABLE 2

| | | Relative units/mg |
|---|---|---|
| Treatment | Time | protein +/− SD^{b} |
| Lipofectin | 48 hours | 30,556 ± 899 |
| 903 (400 nM) | 48 hours | 27,850 ± 2311 |
| 904 (400 nM) | 48 hours | 33,532 ± 3732 |
| 1132 (400 nM) | 48 hours | 30,961 ± 1681 |
| Staurosporine (1 μM) | 6 hours | 264,281 ± 8130 |
| DMSO control for | 6 hours | 34,770 ± 900 |
| staurosporine | | |

^{b}SD = Standard Deviation. All values were assayed in triplicate.

The data in TABLE 2 are consistent with results of immunoblotting tests that show that a single 116 kD PARP protein band was not cleaved in MSN cells following survivin antisense oligonucleotide treatment.

Further experiments were conducted to examine whether survivin antisense oligonucleotide-induced cell death could be blocked by the broad-spectrum caspase inhibitor zVAD-fmk. MSN cells were treated with lipofectin or 600 nM survivin antisense oligonucleotide 904, 906, or mismatch oligonucleotide 1132 alone, or co-treated with 20 μM zVAD-fmk for 48 h, and stained with 0.04% Trypan blue. As shown in FIG. 4B, treatment with survivin antisense oligonucleotide 904 or 906 resulted in 73% and 81%, respectively, Trypan blue positive cells. Upon incubation of survivin antisense oligonucleotide 904 or 906 with zVAD-fmk, 73% and 74%, respectively, of the cells remained Trypan blue positive, indicating that inhibiting caspases did not affect cell death.

Taken together, the caspase-3, PARP, and zVAD-fmk experimental data suggest that survivin antisense oligonucleotide treatment induces cell death in MSN cells in a caspase-independent manner.

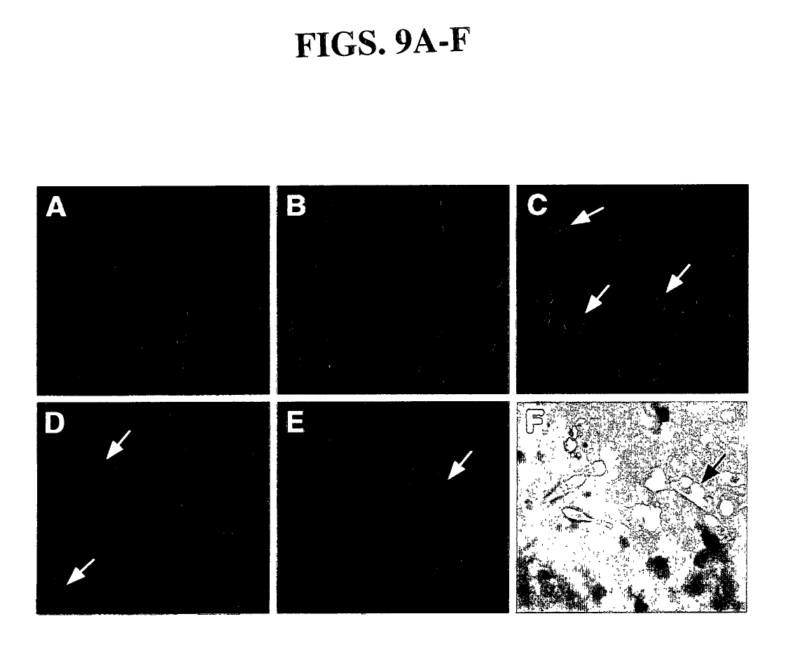

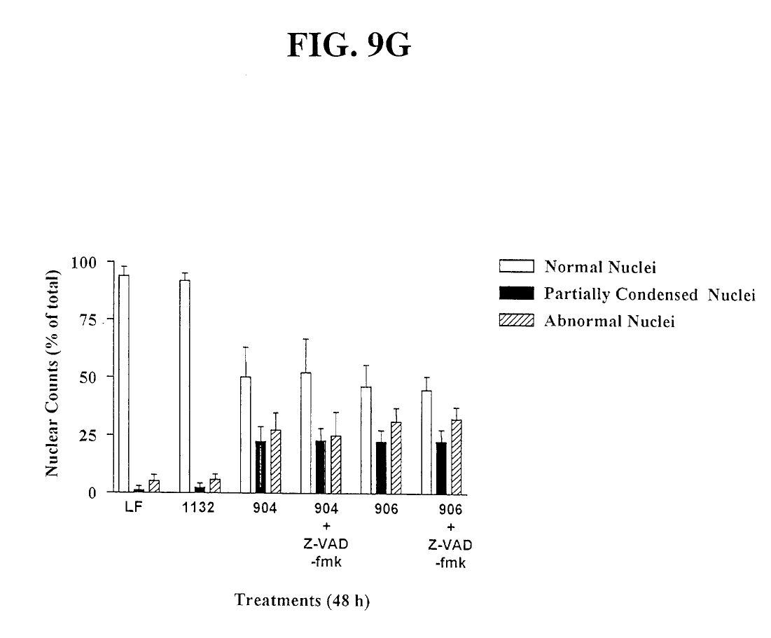

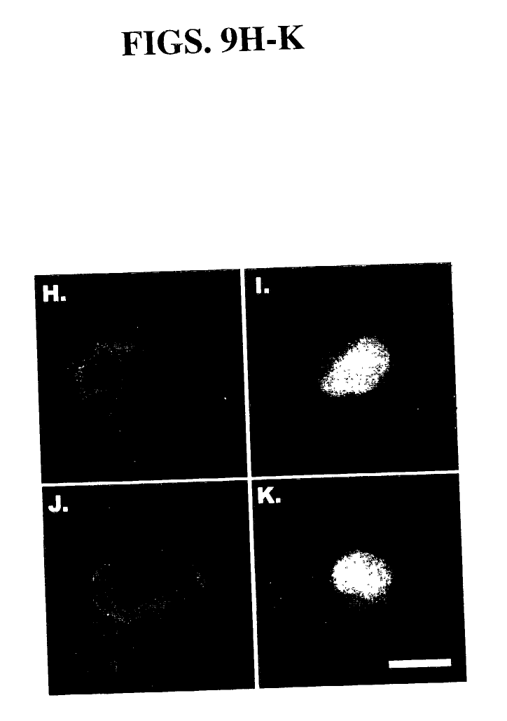

Propidium iodide (PI) staining and phase microscopy were used to assess the nuclear morphology of the survivin antisense oligonucleotide-treated MSN cells. MSN cells were treated with lipofectin (FIG. 9A), or 600 nM mismatch oligonucleotide 1132 (FIG. 9B) or survivin antisense oligonucleotide 904 (FIGS. 9C and 9E) or 906 (FIG. 9D), and stained with PI. To assess the effect on nuclear morphology of co-administration of survivin antisense oligonucleotide and the caspases inhibitor zVAD-fmk, MSN cells were treated with 600 nM survivin antisense oligonucleotide 904, 906, or mismatch oligonucleotide 1132 alone, or in the presence of 20 μM zVAD-fmk, and stained with PI (FIG. 9G).

Lipofectin-treated or mismatch oligonucleotide 1132-treated cells showed normal nuclear morphology (FIGS. 9A and 9B), consistent with our previous observation that at any given time approximately 5% of MSN cells exhibited abnormal nuclei. By contrast, PI staining of survivin antisense oligonucleotide-treated cells revealed a dramatic increase in abnormal appearing nuclei that included multiple multilobulated nuclei (FIGS. 9C and 9E, arrows) and abnormally large nuclei (FIG. 9D), consistent with cells blocked in cell division when the nuclear membrane reassociated. Quantitation following treatment with the survivin antisense oligonucleotides 904 and 906 determined that the percentages of cells with abnormal nuclear morphology were 27% and 31%, respectively, and this percentage was unaltered in cells co-treated with survivin antisense oligonucleotide and zVAD-fmk (FIG. 9G). While no apoptotic bodies or chromatin fragmentation were observed in the survivin antisense oligonucleotide-treated cells, 22% of the survivin antisense oligonucleotide-treated cells contained nuclei with partially condensed chromatin (FIG. 9E, arrowhead; FIG. 9G) and the percentage did not differ with zVAD-fmk treatment. Thus, approximately 50% of the survivin antisense oligonucleotide-treated cells contained abnormal nuclei and condensed chromatin. The partially condensed chromatin is inconsistent with necrotic cell death, and is suggestive of cells undergoing but not completing apoptotic cell death. The observation that the percentage of cell death in the survivin antisense oligonucleotide-treated MSN cells was unaltered in the presence of zVAD-fmk (FIGS. 4B and 9G) further supports that caspase-3 was not activated upon survivin antisense oligonucleotide treatment, and cell death occurred by a caspase-independent mechanism.

These data indicate that apoptotic cell bodies and further condensation of the cell require activated caspases. Experimental results indicate that the existing phenotypes are caspase-independent even upon prolonged (≧72 h) survivin antisense oligonucleotide exposure. In cells undergoing death by a caspase-independent mechanism, apoptosis-inducing factor (AIF) is translocated from the mitochondria to the nucleus prior to cytochrome c release from the mitochondria, and concomitant partial chromatin condensation is observed (Daugas et al. (2000) FASEB J. 14:729-739).

To examine whether down-regulation of survivin induces AIF nuclear translocation, AIF and DAPI double-labeling was performed on MSN cells treated with lipofectin (FIG. 9H), or 600 nM survivin antisense oligonucleotide 904 (FIG. 9I), mismatch oligonucleotide 1132 (FIG. 9J), or survivin antisense oligonucleotide 906 (FIG. 9K). As shown in FIGS. 9H and 9J, both lipofectin-treated and mismatch oligonucleotide 1132-treated cells show robust AIF staining in the cytosol. Only 4-8% of the cells contained nuclear localization of AIF, and DAPI staining showed that 95% of the cells had normal-appearing nuclei.

By contrast, 45-51% of survivin antisense oligonucleotide 904- and 906-treated cells showed AIF nuclear translocation (FIGS. 9I and 9K). DAPI staining indicated that 37% of these survivin antisense oligonucleotide-treated cells contained partially condensed chromatin, and 25% had abnormal nuclei similar to the PI staining in FIG. 9G. The lack of highly condensed apoptotic bodies, the nuclear translocation of AIF, and the morphologic appearance of the nuclei by PI and DAPI are consistent with cells undergoing cell death by a caspase-independent mechanism. Thus, the combined PI, DAPI, and AIF data are consistent with survivin antisense oligonucleotide treatment causing a disruption in the cell cycle, likely mitotic catastrophe, resulting in cell death.

Other experiments were conducted to evaluate the inactivity of caspase-3 and lack of PARP cleavage following treatment of the MSN cells. It was postulated that another member of the inhibitor-of-apoptosis (IAP) family of proteins was activated and effectively blocked PARP cleavage during the survivin antisense treatment. XIAP, another member of the IAP family, was therefore examined by immunoblotting following survivin antisense treatment in both MSN and TC620 cells.

MSN cells were treated with lipofectin or 400 nM survivin antisense oligonucleotides 900, 901, 903, 904, 905 and 906. Total protein was isolated 48 hours after treatment, and 100 μg of protein was loaded per lane. The blot was incubated with a monoclonal antibody against XIAP (1:1000; IgG₁). Visualization was by enhanced chemiluminescence (ECL). To confirm equal loading, blots were stripped, re-exposed to ECL to confirm that the antibody was removed, and incubated with β-tubulin monoclonal antibody (1:1000). The experiment was performed twice. With survivin antisense oligonucleotide treatment, the fold change of XIAP protein over β-tubulin relative to lipofectin was: 900, 2.3; 901, 3.9; 903, 5.2; 904, 8.0; 905, 7.6; 906, 8.3.

TC620 oligodendroglioma cells were transfected with lipofectin or increasing concentrations of survivin antisense oligonucleotide 904 (25 to 400 nM). Total protein was isolated after 48 hours of treatment and 75 μg of total protein was loaded per lane. The experiment was performed once. The fold change of XIAP protein over β-tubulin relative to lipofectin upon treatment with survivin antisense oligonucleotide 904 was: 25 nM, 1.3; 50 nM, 1.0; 100 nM, 0.71; 200 nM, 0.67; 400 nM, 0.66.

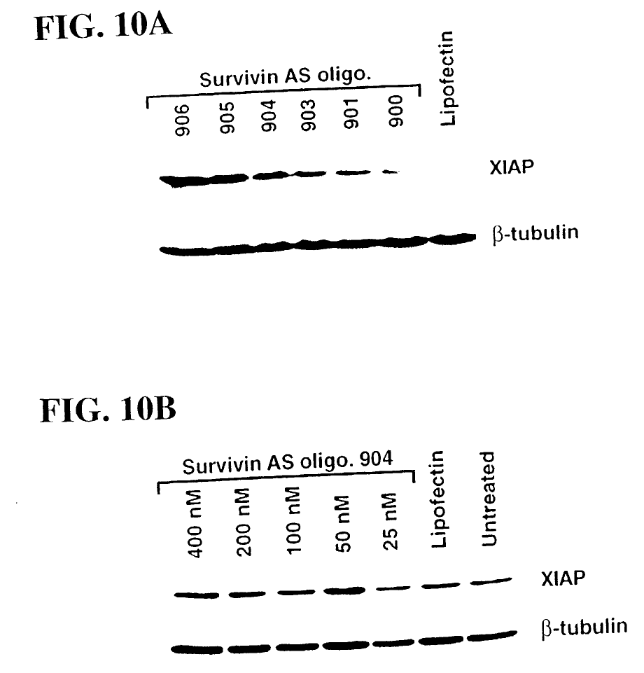

As shown in FIG. 10A, an eight-fold increase in XIAP was observed in MSN cells 48 hours following treatment with survivin antisense oligonucleotide 904, while the lipofectin-treated MSN cells had low XIAP levels. In TC620 cells, treatment with survivin antisense oligonucleotide 904 did not induce any increase in the XIAP protein levels relative to lipofectin treatment, yet apoptosis still occurred (FIG. 10B).

The results shown in FIG. 10B suggest that an increase of XIAP observed in MSN cells may account for the inhibition of caspase-3 activity. They further suggest that a caspase-3-dependent mechanism of cell death occurs in the survivin antisense treated TC620 cells, while in MSN cells, survivin inhibition leads to cell death by a caspase-3-independent mechanism.

The above results indicate that the treatment of either the MSN neuroblastoma or TC620 oligodendroglioma cells with G₂/M phase blockers showed an up-regulation of survivin protein levels suggestive of the ability of survivin to facilitate the transition of tumor cells through the G₂/M checkpoint into mitosis. During interphase, survivin is associated with y-tubulin around spindle centrioles, while during metaphase and anaphase, survivin is associated with microtubules of the mitotic spindle (Li et al. (1998) Nature 396:580-584). Although the exact mechanism for how survivin regulates cell cycle phase transition and mitotic progression is not presently known, the expression of survivin probably functions to enhance tumor cell progression through the cell cycle.

While survivin antisense oligonucleotide treatment results in cell death for both the MSN neuroblastoma and TC620 oligodendroglioma cell lines, the cell death pathway was shown to be markedly different. Previously, it had been shown that survivin inhibition for 48-64 hours increases active caspase-3 in HeLa cells (Li et al. (1999) Nat. Cell. Biol. 1:461-466) and lung adenocarcinoma cells (Olie et al. (2000) Cancer. Res. 60:2805-2809). In MSN cells, however, antisense oligonucleotides 903 and 904 did not activate caspase-3 or cleave the caspase-3 substrate, PARP, suggesting that these cells undergo cell death by a pathway that is independent of caspase-3 activation. Although not to be bound by this theory, it is believed that the MSN cells suppressed caspase-3 activation by the activation of other caspase-3 binding proteins. This finding is supported by recent studies which have demonstrated that other members of the IAP family of proteins (XIAP, c-IAP-1 and c-IAP-2) have the ability to interact directly with caspases and inhibit their ability to cleave substrates (Deveraux et al. (1997) Nature 388:300-304; Roy et al. (1997) EMBO J. 16:6914-6925).

To test this theory, another IAP family protein, XIAP, was examined and it was determined that treatment of MSN cells with survivin antisense oligonucleotides was accompanied by a dramatic increase in XIAP. This suggests that MSN cells have the ability to up-regulate other IAP family members to compensate for the decrease or the loss of a key anti-apoptotic protein like survivin. The fact that MSN cells still underwent cell death indicates that survivin has more than one function in the cell and the possible inhibition of caspase-3 cannot overcome the functional role of survivin in MSN cells.

In contrast to the neuroblastoma cell line, the oligodendroglioma cell line TC620 underwent a caspase-3-dependent apoptotic cell death as assessed by PARP cleavage. Treatment of TC620 cells with increasing concentrations of the survivin antisense oligonucleotide 904 resulted in the cleavage of the 116 kD caspase-3 substrate PARP to generate the 85 kD cleaved fragment. Treatment of TC620 cells with survivin antisense oligonucleotides did not alter XIAP and the TC620 cells proceeded to undergo cell death in a caspase-3-dependent manner. Thus, these results indicate that inhibition of survivin expression by survivin antisense oligonucleotides unequivocally induced cell death in both the neuroblastoma and oligodendroglioma cell lines.

However, the induction or inhibition of caspase activation upon inhibition of survivin expression appears to be contingent upon the ability of each cell type to regulate and alter the levels of other IAP family members, particularly XIAP. This in part also supports the observation that survivin binds quantitatively in vitro to an IAP-inhibiting protein Smac/DIABLO (Du et al. (2000) Cell 102:33-42), raising the possibility that it might suppress caspases indirectly by freeing other IAP family proteins from the constraints of this protein.

While not to be bound by any particular theory, the studies described herein suggest that survivin functions predominantly in cell division. Since cell cycle progression is universal, it is consistent with this theory that survivin expression was observed in all neural tumor cells examined. Survivin null mice die in embryogenesis, are polyploid, and have disrupted microtubules consistent with a role in cell division (Uren et al. (2000) Curr. Biol. 10:1319-1328). Proteins involved in control of chromosome number or ploidy have been implicated in regulating programmed cell death, and survivin may have developed IAP function via its BIR domain to aid in normal cell division and survival. In tumor cells, the death program is often compromised and regulated abnormally by a process of random mutation and selection, becoming progressively more malignant as they accumulate mutations that improve their ability to survive and proliferate. Thus, it appears that the role of survivin in cell division is co-opted by tumor cells to aid in their survival.

The synthetic survivin-specific oligonucleotides of the invention are also useful for various methods which the invention also provides. The invention provides a method of enhancing apoptosis in a cancer cell expressing survivin. The invention also provides a method of inhibiting the synthesis of survivin in a cell that expresses functional survivin, comprising contacting the cell with an oligonucleotide of the invention, as described above. Further, the invention also provides a method of inhibiting the growth of a neoplastic cell expressing survivin, comprising contacting the cell with an oligonucleotide of the invention, as described above. In some embodiments, the neoplastic cell is a nervous system cancer cell, such as a brain cancer cell.

In these methods, the synthetic oligonucleotides of the present invention are contacted with a cancer cell. These synthetic oligonucleotides are complementary to a nucleic acid encoding survivin protein. In some instances, the synthetic oligonucleotides have a nucleic acid sequence selected from the group consisting of SEQ ID NOS: 1, 2, 3, 4, 5, 6, 7, 8, 9, 10, and 11. In other instances, the synthetic oligonucleotides have a nucleic acid sequence that is at least 85% identical to a nucleic acid sequence selected from the group consisting of SEQ ID NOS: 1, 2, 3, 4, 5, 6, 7, 8, 9, 10, and 11. In still other instances, the percent identity is at least 90%, for example, at least 93%, or at least 95% identical to a nucleic acid sequence selected from the group consisting of SEQ ID NOS: 1, 2, 3, 4, 5, 6, 7, 8, 9, 10, and 11. In yet other instances, the percent identity is at least 97%, for example, at least 98%, or at least 99% identical to a nucleic acid sequence selected from the group consisting of SEQ ID NOS: 1, 2, 3, 4, 5, 6, 7, 8, 9, 10, and 11.

In such methods, the synthetic oligonucleotides of the invention are “antisense” oligonucleotides which are isolated and which specifically hybridize under cellular conditions, with the cellular survivin mRNA so as to inhibit expression of the encoded survivin protein, e.g., by inhibiting transcription and/or translation. Generally, the binding may be by conventional base pair complementarity, or, for example, in the case of binding to DNA duplexes, through specific interactions in the major groove of the double helix. In general, use of the antisense oligonucleotides refers to the range of techniques generally employed in the art, and includes any method which relies on specific binding to oligonucleotide sequences.

As discussed above, exemplary nucleic acid molecules for use as antisense oligonucleotides are phosphoramidate, phosphorothioate, and methylphosphonate analogs of DNA. Additionally, general approaches to constructing oligomers useful in antisense therapy have been reviewed, for example, by Van der Krol et al. (1988) Biotechniques 6:958-976; and Stein et al. (1988) Cancer Res. 48:2659-2668. Accordingly, the modified oligomers of the invention are useful in therapeutic, diagnostic, and research contexts.

In addition, the oligonucleotides of the invention may be used as diagnostic reagents to detect the presence or absence of the target DNA or RNA sequences to which they specifically bind. The antisense constructs of the present invention, by down-regulating the expression of survivin protein, can be used in the manipulation of tissue, both in vivo and ex vivo tissue cultures.

An antisense construct of the present invention can be delivered, for example, as an expression plasmid which, when transcribed in the cell, produces RNA which is complementary to at least a unique portion of the cellular mRNA which encodes survivin protein. Alternatively, the antisense construct is an oligonucleotide probe which is generated ex vivo and which, when introduced into the cell, causes inhibition of expression by hybridizing with survivin mRNA. Such oligonucleotide probes are in some embodiments modified oligonucleotides which are resistant to endogenous nucleases, e.g., exonucleases and/or endonucleases, and are therefore stable in vivo.

The oligonucleotides of the invention, when in the form of a therapeutic formulation, are also useful in treating diseases, disorders, and conditions associated with cancer. In such methods, a therapeutic amount of a synthetic oligonucleotide of the invention effective in inhibiting the expression of survivin, in some instances with another antitumor agent, is administered to a cell. This cell may be part of a cell culture or a tissue culture, or may be part or the whole body of an animal such as a human or other mammal.

If the cells to be treated by the methods of the invention are in an animal, the oligonucleotides of the invention (and any additional anticancer agents, if part of the therapeutic methods) are administered by conventional procedures as therapeutic compositions in pharmaceutically acceptable carriers. For example, cisplatin and its analogs, as well as other platinum compounds, taxol, taxotere, adriamycin, camptosar (e.g., CPT-11), C225, topotecan, 5-fluorouracil, and their respective analogs, and cytotoxins can be administered to cancer patients as described by Slapak et al. in Harrison's Principles of Internal Medicine, 14^{th} Edition, McGraw-Hill, N.Y. (1998) Chapter 86.

The characteristics of the carrier will depend on the route of administration, as described below. Such a composition may contain, in addition to the synthetic oligonucleotide and carrier, diluents, fillers, salts, buffers, stabilizers, solubilizers, and other materials well known in the art. The pharmaceutical composition of the invention may also contain other active factors and/or agents which enhance inhibition of survivin gene or mRNA expression or which will reduce cancer cell proliferation. For example, combinations of synthetic oligonucleotides, each of which is directed to different regions of survivin nucleic acid may be used in the pharmaceutical compositions of the invention.

The pharmaceutical composition of the invention may further contain nucleoside analogs such as azidothymidine, dideoxycytidine, dideoxyinosine, and the like. Such additional factors and/or agents may be included in the pharmaceutical composition to produce a synergistic effect with the synthetic oligonucleotide of the invention, or to minimize side-effects caused by the synthetic oligonucleotide of the invention. Conversely, the synthetic oligonucleotide of the invention may be included in formulations of a particular anti-survivin gene or gene product factor and/or agent to minimize side effects of the anti-survivin gene factor and/or agent.

The pharmaceutical composition of the invention may be in the form of a liposome in which a synthetic oligonucleotide of the invention is combined, in addition to other pharmaceutically acceptable carriers, with amphipathic agents, such as lipids, which exist in aggregated form as micelles, insoluble monolayers, liquid crystals, or lamellar layers which are in aqueous solution. Suitable lipids for liposomal formulation include, without limitation, monoglycerides, diglycerides, sulfatides, lysolecithin, phospholipids, saponin, bile acids, and the like. One particularly useful lipid carrier is lipofectin. Preparation of such liposomal formulations is conventional in the art, as disclosed, for example, in U.S. Pat. Nos. 4,235,871, 4,501,728, 4,837,028, and 4,737,323. The pharmaceutical composition of the invention may further include compounds such as cyclodextrins and the like which enhance delivery of oligonucleotides into cells, as described by Zhao et al. Antisense Research & Development 5:185-192 (1995), or slow release polymers.

As used herein, the term “therapeutically effective amount” means the total amount of each active component of the pharmaceutical composition or method that is sufficient to show a meaningful patient benefit, i.e., reducing the size of a tumor or inhibiting its growth or inhibiting the proliferation rate of cancer cells. When applied to an individual active ingredient, administered alone, the term refers to that ingredient alone. When applied to a combination, the term refers to combined amounts of the active ingredients that result in the therapeutic effect, whether administered in combination, serially or simultaneously. The terms “therapeutically effective amount” and “therapeutically effective period of time” are used to denote known treatments at dosages and for periods of time effective to reduce neoplastic cell growth.

In practicing the method of treatment or use of the present invention, a therapeutically effective amount of one, two, or more of the synthetic oligonucleotides of the invention is administered to a subject afflicted with a disease or disorder related to cancer. The synthetic oligonucleotide of the invention may be administered in accordance with the method of the invention either alone or in combination with various anticancer agents such as, but not limited to, oxidizing agents or cytotoxins, and/or other known therapies for cancer. When co-administered with one or more other therapies, the synthetic oligonucleotide of the invention may be administered either simultaneously with the other treatment(s), or sequentially. If administered sequentially, the attending physician will decide on the appropriate sequence of administering the synthetic oligonucleotide of the invention in combination with the other therapy.

Administration of the synthetic oligonucleotide of the invention used in the pharmaceutical composition or to practice the method of the present invention can be carried out in a variety of conventional ways, such as intraocular administration, oral ingestion, inhalation, or cutaneous, subcutaneous, intramuscular, or intravenous injection. Administration may be bolus, intermittent, or continuous, depending on the condition and response, as determined by those with skill in the art. In some embodiments of the methods of the invention described above, the oligonucleotide is administered locally (e.g., intraocularly or interlesionally) and/or systemically. The term “local administration” refers to delivery to a defined area or region of the body, while the term “systemic administration” is meant to encompass delivery to the whole organism by oral ingestion, or by intramuscular, intravenous, subcutaneous, or intraperitoneal injection.

When a therapeutically effective amount of synthetic oligonucleotide of the invention is administered orally, the synthetic oligonucleotide will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5% to 95% synthetic oligonucleotide, and in some embodiments from about 25% to 90% synthetic oligonucleotide. When administered in liquid form, a liquid carrier such as water, petroleum, oils of animal or plant origin such as peanut oil, mineral oil, soybean oil, sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5% to about 90% by weight of the synthetic oligonucleotide, and in some embodiments from about 1% to about 50% synthetic oligonucleotide.

When a therapeutically effective amount of synthetic oligonucleotide of the invention is administered by intravenous, subcutaneous, intramuscular, intraocular, or intraperitoneal injection, the synthetic oligonucleotide will be in the form of a pyrogen-free, parenterally acceptable aqueous solution. The preparation of such parenterally acceptable solutions, having due regard to pH, isotonicity, stability, and the like, is within the skill in the art. In at least some embodiments, the pharmaceutical composition for intravenous, subcutaneous, intramuscular, intraperitoneal, or intraocular injection should contain, in addition to the synthetic oligonucleotide, an isotonic vehicle such as Sodium Chloride Injection, Ringer's Injection, Dextrose Injection, Dextrose and Sodium Chloride Injection, Lactated Ringer's Injection, or other vehicles as known in the art. The pharmaceutical composition of the present invention may also contain stabilizers, preservatives, buffers, antioxidants, or other additives known to those of skill in the art.

The amount of synthetic oligonucleotide in the pharmaceutical composition of the present invention will depend upon the nature and severity of the condition being treated, and on the nature of prior treatments that the patient has undergone. Ultimately, the attending physician will decide the amount of synthetic oligonucleotide with which to treat each individual patient. Initially, the attending physician will administer low doses of the synthetic oligonucleotide and observe the patient's response. Larger doses of synthetic oligonucleotide may be administered until the optimal therapeutic effect is obtained for the patient, and at that point the dosage is not increased further. It is contemplated that the various pharmaceutical compositions used to practice the method of the present invention should contain about 10 μg to about 20 mg of synthetic oligonucleotide per kg body or organ weight.

The duration of intravenous therapy using the pharmaceutical composition of the present invention will vary, depending on the severity of the cell proliferation disorder being treated and the condition and potential idiosyncratic response of each individual patient. Ultimately the attending physician will decide on the appropriate duration of intravenous therapy using the pharmaceutical composition of the present invention.

If oligonucleotides of the invention are administered locoregionally (e.g., intraperitoneally) as opposed to systemically, normal tissue uptake should be reduced. In addition, methods of encapsulating oligonucleotides in liposomes and targeting these liposomes to selected tissues by inserting proteins into the liposome surface are now conventional.

Standard reference works setting forth the general principles of the genetic and molecular biology technology described herein include Ott and Hoh (2000) Am. J. Hum. Genet. 67:289-294; Zubay G. (1987) Genetics (The Benjamin/Cummings Publishing Co., Menlo Park, Calif.); Ausubel et al. (1999) Current Protocols in Molecular Biology (John Wiley & Sons, New York, N.Y.); Sambrook et al. (1989) Molecular Cloning: A Laboratory Manual, 2d Ed. (Cold Spring Harbor Laboratory Press, Plainview, N.Y.); Kaufman et al., Eds.(1995) Handbook of Molecular and Cellular Methods in Biology and Medicine (CRC Press, Boca Raton, La.); and McPherson, Ed. (1991) Directed Mutagenesis: A Practical Approach (IRL Press, Oxford). Standard reference works setting forth the general principles of immunology and inflammation include Gallin et al. (1988) Inflammation: Basic Principles and Clinical Correlates (Raven Press, New York, N.Y.); Kuby, J. (1997) Immunology, 3^{rd} ed., (W. H. Freeman, New York, N.Y.); Coligan et al., Eds. (1991) Current Protocols in Immunology (John Wiley & Sons, New York, N.Y.); and Hurley (1983) Acute Inflammation, 2^{nd} ed. (Churchill Livingstone, N.Y.).

The following examples are intended to further illustrate certain embodiments of the invention and are not limiting in nature. Those skilled in the art will recognize, or be able to ascertain, using no more than routine experimentation, numerous equivalents to the specific substances and procedures described herein. Such equivalents are considered to be within the scope of this invention, and are covered by the following claims.

### EXAMPLES

1. Synthesis of Antisense Oligonucleotides

Antisense oligonucleotides targeting survivin mRNA (GenBank accession number U75285) were designed based on the selection criteria described earlier (Agrawal and Kandimalla, Mol. Med. Today (2000) 6:72-81). Synthesis of 20-mer phosphorothioate or mixed backbone modified antisense oligonucleotides targeting different regions of the human survivin mRNA was performed using standard procedures (see, e.g., Agrawal (1997) Proc. Natl. Acad. Sci. (USA) 94:2620-2625). The identity and purity of the oligonucleotides were confirmed by conventional ³¹p nuclear magnetic resonance, capillary gel electrophoresis, hybridization melting temperature, A₂₆₉, and MALDI/TOF mass ratio spectral analysis (see, e.g., Agrawal (1997) Proc. Natl. Acad. Sci. (USA) 94:2620-2625).

2. Cell Culture

The human neuroblastoma cell line, MSN (Reynolds et al. (1986) J. Natl. Cancer. Inst. 76:375-387), was grown in RPMI 1640 supplemented with 23.8 mM sodium bicarbonate, 10% fetal calf serum, 0.1 mM non-essential amino acids (GIBCO, Grand Island, N.Y.), 0.47 mM L-serine, and 0.38 mM L-asparagine. The oligodendroglioma cell lines, HOG and TC620, obtained from Dr. Anthony Campagnoni at the University of California Los Angeles, USA, were maintained in Iscove's medium (Fisher Scientific, Pittsburgh, Pa.) plus 10% fetal calf serum. The glioblastoma cell line, ATCC No. HTB 14 and the astrocytoma cell line, ATCC No. HTB 17, were obtained from American Type Culture Collection, Manassas, Va., and were maintained in DMEM plus 10% fetal calf serum. Previously shown to express survivin, Jurkat cells (obtained from Dr. Marshall Horwitz, Albert Einstein College of Medicine, Bronx, N.Y.) served as a positive control for immunoblotting (Conway et al. (2000) Blood 95:1435-1442). Jurkat cells were grown in RPMI 1640 plus 10% fetal calf serum. Cells were grown in a humidified atmosphere containing 5% (HOG, TC620) or 8% (MSN, ATCC No. HTB 14, and ATCC No. HTB 17) CO₂ at 37° C.

3. RNA Isolation and Northern Blot Analysis

Total RNA was isolated from the cell lines using TRI-reagent (Molecular Research Center, Cincinnati, Ohio.). Northern blot analysis was performed as previously described (Shafit-Zagardo et al. (1988) J. Neurochem. 51:1258-1266). Nitrocellulose blots containing 30 μg of total RNA was hybridized with a human survivin cDNA or a cDNA to 18 S RNA. The probes were labeled using [α-³²P] dCTP and the Multiprime DNA labeling system (Amersham, Arlington Heights, Ill.).

4. Nocodazole, Taxotere and Vinblastine Treatment

To block cells in G₂/Ml MSN cells or TC620 cells were treated with 10 μM nocodazole, 1 μM taxotere, or 250 nM vinblastine for 16 hours, at which time total protein homogenates were isolated and proteins were examined following sodium dodecyl sulfate-polyacrylamide gel electrophoresis (SDS-PAGE) and immunoblotting.

5. Oligonucleotide Transfections

Eleven different antisense phosphorothioate oligonucleotides to the survivin gene (GenBank accession number U75285) and two mismatched phosphorothioate oligonucleotides were designed based on the selection criteria described earlier (Agrawal et al. (2000) Mol. Med. Today 6:72-81).

The antisense oligonucleotides were synthesized on solid support with an automated DNA synthesizer using β-cyanoethylphosphoramidite chemistry. In order to prevent rapid degradation of the antisense oligonucleotides by cellular nucleases, oxidation was carried out with Becauge sulfurizing agent to obtain phosphorothioate backbone modified oligonucleotides. After their synthesis, the oligonucleotides were released from the solid support, deprotected, purified by reverse-phase HPLC, desalted, filtered and lyophilized. The purity and sequence integrity of oligonucleotides was ascertained by capillary gel electrophoresis and MALDI-TOF mass spectral analysis and the concentrations measured at 260 nM. Cells were grown in 100 mm dishes and oligonucleotide treatment was performed on subconfluent cultures in the presence of Lipofectin (GIBCO) according to the manufacturer. Forty-eight hours following treatment, total protein homogenates were isolated.

6. Protein Preparation and Immunoblotting

Total protein homogenates were prepared according to a previously described procedure (Albala et al. (1995) J. Neurochem. 64:2480-2490). Protein content was measured using the Bio-Rad detection system (Bio-Rad Laboratories, Hercules, Calif.). Equal amounts of protein were analyzed by SDS-PAGE on 10% gels (Laemmli (1970) Nature 227:680-685). The resolved polypeptides were electrophoretically transferred to nitrocellulose (Towbin et al. (1979) Proc. Natl. Acad. Sci. USA 76:4350-4354).

Human survivin was expressed as a GST-fusion protein and subsequently used to absorb the survivin polyclonal antibody to demonstrate specificity during immunoblotting. Ten micrograms of the fusion protein was incubated with the survivin antibody (1:500) at 4° C. for 2 hours to overnight prior to immunoblotting. Immunoblots were routinely blocked with 5% non-fat, dry milk in 1×TBS (0.14M NaCl, 0.001 M Tris Base, pH 7.4). Blots were cut at 32.9 kD, and the bottom part of the blots were incubated with the survivin antibody overnight at 4° C. and visualized by enhanced chemiluminescence (Enhanced Chemiluminescence, Amersham, Arlington Heights, Ill.) as previously described (Sharma et al. Cell. Motil. Cytoskeleton 27:234-247).

The top part of the blots that contained proteins higher than 32.9 kD were incubated with a tubulin antibody at room temperature for 2 hours. Survivin polyclonal antibody was purchased from R & D Systems, Inc. (Minneapolis, Minn.). A second polyclonal antibody yielded identical results (Alpha Diagnostics International, San Antonio, Tex.). A generic β-tubulin monoclonal antibody (TUB 2.1) was purchased from SIGMA (St. Louis, Mo.). The XIAP mAb was purchased from StressGen Biotechnologies Corp. (Victoria, Canada). The poly-ADP ribosyl polymerase (PARP) mAb was purchased from PharMingen/Transduction Laboratories (San Diego, Calif.).

7. Trypan Blue Assay

Cells were harvested 48 hours after survivin antisense treatment, stained with 0.04% Trypan blue (GIBCO), and counted on a hemocytometer. The number of Trypan blue positive cells relative to the total number of cells in each field was obtained and the data were expressed as a percentage of dead cells relative to the total cell number. Individual experiments were performed in triplicate. MSN cell studies were performed three times, while studies in TC620 cells were performed once.

8. Caspase-3 Activity Assay

MSN cells were treated with lipofectin, 400 nM survivin antisense oligonucleotide 903, 904, or mismatched oligonucleotide 1132, for a 48 hour period. Cell pellets were washed twice in cold PBS and re-suspended in ice-cold hypotonic cell lysis buffer (25 mM HEPES, pH 7.5, 5 mM MgCl₂, 5 mM EDTA, 5 mM DTT, 2 mM PMSF, 10 μg/ml pepstatin A, 10 μg/ml leupeptin). Re-suspended pellets were incubated on ice for 20 min, followed by brief sonication for two seconds. Lysates were centrifuged at 14,000 rpm for 20 minutes at 4° C. The supernatant was retained and the protein concentration analyzed by the Bio-Rad protein assay described above.

Supernatants were assayed in triplicate, with and without the caspase-3 inhibitor, Ac-DEVD-CHO, using black opaque, 96 well, flat bottom plates (Greiner Laboratories, USA Scientific Inc., Orlando, Fla.). 50-100 μg of the supernatant was incubated in caspase-3 assay buffer (100 mM HEPES, pH 7.5, 10% sucrose, 0.1% CHAPS and 10 mM DTT) with either 2 μl of DMSO or 2 μl of 2.5 mM Ac-DEVD-CHO (Pharmingen, San Diego, Calif.) so that the final concentration of the inhibitor was 50 μM in a final volume of 100 μl. It was then incubated at 30° C. for 30 minutes. Subsequently, 2 μl of the caspase-3 substrate 2.5 mM Ac-DEVD-AMC (Pharmingen, San Diego, Calif.) (final concentration 50 μM)) was added to each well. Plates were incubated at 30° ° C. for 60 minutes in the dark.

Fluorescence of the reaction was measured using a SPECTRAmax GEMINI spectrofluorometer using SOFTmax® PRO software (Molecular Devices, Sunnyvale, Calif.) at an excitation wavelength of 355 nm and an emission wavelength of 460 nm with a cutoff filter of 455 nm. The data is presented as the ratio of the mean relative fluorescence units/mg protein±SD. All experiments included the caspase-3 inhibitor Ac-DEVD-CHO that eliminated caspase-3 activation.

9. Propidium Iodide (PI) Staining

Survivin antisense oligonucleotide-treated cells were fixed with 4% paraformaldehyde for 30 min at room temperature, washed with 1×TBS, permeabilized with 0.1% Triton X-100 for 30 min and treated with 10 μg/ml DNasc free RNase A (Sigma) for 60 min. Nuclei were stained with 200 μg /ml PI for 30 min at 4° C. and washed twice with 1×TBS. Nuclear morphology was assessed on an inverted Olympus 1×70 fluorescence microscope equipped with phase and epifluorescence optics. For each treatment about 600 nuclei were scored as normal, condensed (apoptotic), or abnormal (macronuclei, multilobed) on 15 random, 40×objective fields in duplicate. Quantification results are mean±SEM obtained from two independent experiments performed in duplicate.

10. TUNEL Assay

The TUNEL assay was performed to assess apoptotic cell death in survivin antisense oligonucleotide-treated TC620 cells using the In Situ Cell Death Detection Kit, Fluorescein (Roche Molecular Biochemicals, Indianapolis, Ind.). The TUNEL reaction preferentially labels cleaved genomic DNA generated during apoptosis, by the addition of fluorescein dUTP at strand breaks. Lipofectin, mismatch oligonucleotide 1132 or survivin antisense oligonucleotide-treated TC620 cells were fixed and permeabilized as described for PI staining. Cells were washed and incubated in the TUNEL reaction mixture, prepared according to the manufacture's recommendations, for 1 h at 37° C. Omission of the terminal deoxynucleotidyl transferase in the label solution served as a negative control for the TUNEL fluorescence staining. Cells were washed twice and counterstained with the DNA specific dye DAPI (1:1000 of a 1 mg/ml stock; 15 min at room temperature). Cells were examined with an Olympus 1×70 inverted microscope. For each treatment 15 random, 40×objective fields consisting of about 1000 cells were examined in duplicate chamber slides. TUNEL-positive nuclei were expressed as a percent of the total number of cells per individual field.

11. Apoptosis-Inducing Factor (AIF) Immunostaining and Quantitation

Lipofectin, mismatch oligonucleotide 1132 or survivin antisense oligonucleotide-treated MSN cells were fixed and permeabilized as described for PI staining, and blocked for 1 h at room temperature with 10% normal goat serum in 5% non-fat, dry milk in 1×TBS. The cells were incubated with an AIF polyclonal antibody (1:100, Santa Cruz Biotechnology Inc., Santa Cruz, Calif.) overnight at 4° C., and revealed with a goat anti-rabbit IgG conjugated to TRITC (Southern Biotechnology Associates, Birmingham, Ala.). Omission of the primary antibody confirmed that the immunostaining was specific. Cells were counterstained with the DNA-specific dye DAPI (1:1000 of a 1 mg/ml stock; 15 min at room temperature). Cells were examined with an Olympus 1×70 inverted microscope. Fluorescent images were collected using a 12-bit Photometrics cooled CCD camera. For each treatment, 15 random, 40×objective fields consisting of about 600 cells were examined. TRITC (red) and DAPI (blue) stained cells were scored as having AIF staining either in the mitochondria or the nucleus relative to the total number examined. In parallel, the DAPI stained nuclei were also scored as normal, condensed, or abnormal nuclei. Experiments were performed in duplicate.

### EQUIVALENTS

As will be apparent to those skilled in the art to which the invention pertains, the present invention may be embodied in forms other than those specifically disclosed above without departing from the spirit or essential characteristics of the invention. The particular embodiments of the invention described above are, therefore, to be considered as illustrative and not restrictive. The scope of the invention is as set forth in the appended claims rather than being limited to the examples contained in the foregoing description.

What is claimed is:

1. A synthetic oligonucleotide complementary to a nucleic acid encoding survivin protein, the synthetic oligonucleotide having SEQ ID NO: 1, 2, 3, 4, 5, 6, 7, 8, 9, 10, or 11.

2. The oligonucleotide of claim 1 having phosphorothioate internucleoside linkages.

3. The oligonucleotide of claim 1 having SEQ ID NO: 6, 7, or 9.

4. The oligonucleotide of claim 3 having phosphorothioate internucleoside linkages.

5. A method of inhibiting the synthesis of survivin in a cell that expresses survivin, comprising contacting the cell with an oligonucleotide of claim 1.

6. A method of inhibiting the growth of a cancer cell expressing survivin protein, comprising contacting the cell with an oligonucleotide of claim 1.

7. The method of claim 6, wherein the cancer cell is a nervous system cancer cell.

8. The method of claim 7, wherein the cancer cell is a neuroblastoma cell or an oligodendroglioma cell.

9. A method of enhancing apoptosis in a cell expressing survivin, comprising contacting the cell with an oligonucleotide of claim 1.

10. The method of claim 9, wherein the cell is a cancer cell.

11. The method of claim 10, wherein the cancer cell is a nervous system cancer cell.

12. The method of claim 11, wherein the nervous system cancer cell is a neuroblastoma cell or an oligodendroglioma cell.

13. A pharmaceutical composition comprising the synthetic oligonucleotide of claim 1 and a pharmaceutically acceptable carrier.

14. A method for treating a nervous system tumor in a mammal, comprising administering to the mammal a therapeutically effective amount of the pharmaceutical composition of claim 13.

15. A synthetic oligonucleotide complementary to a nucleic acid encoding survivin protein, the synthetic oligonucleotide having a nucleic acid sequence that is at least 85% identical to the nucleic acid sequence of SEQ ID NO: 1, 2, 3, 4, 5, 6, 7, 8, 9, 10, or 11.

16. The oligonucleotide of claim 15 having phosphorothioate internucleoside linkages.

17. The oligonucleotide of claim 15, having a nucleic acid sequence that is at least 85% identical to the nucleic acid sequence of SEQ ID NO: 6, 7, or 9.

18. The oligonucleotide of claim 17 having phosphorothioate internucleoside linkages.

19. A method of inhibiting the synthesis of survivin in a cell that expresses survivin, comprising contacting the cell with an oligonucleotide of claim 15.

20. A method of inhibiting the growth of a cancer cell expressing survivin protein, comprising contacting the cell with an oligonucleotide of claim 15.

21. The method of claim 20, wherein the cancer cell is a nervous system cancer cell.

22. The method of claim 21, wherein the cancer cell is a neuroblastoma cell or an oligodendroglioma cell.

23. A method of enhancing apoptosis in a cell expressing survivin, comprising contacting the cell with an oligonucleotide of claim 15.

24. The method of claim 23, wherein the cell is a cancer cell.

25. The method of claim 24, wherein the cancer cell is a nervous system cancer cell.

26. The method of claim 25, wherein the nervous system cancer cell is a neuroblastoma cell or an oligodendroglioma cell.

27. A pharmaceutical composition comprising the synthetic oligonucleotide of claim 15 and a pharmaceutically acceptable carrier.

28. A method for treating a nervous system tumor in a mammal, comprising administering to the mammal a therapeutically effective amount of the pharmaceutical composition of claim 27.
